(19) United States
(12) Patent Application Publication
(10) Pub. No.: US 2015/0216471 A1
(43) Pub. Date: Aug. 6, 2015

(54) DIAGNOSTIC DEVICE AND METHOD FOR SENSING HYDRATION STATE OF A MAMMALIAN SUBJECT

(71) Applicant: HYDRADX, INC., Altadena, CA (US)

(72) Inventors: Andrew S. Goldstein, Portland, OR (US); Kirk Charles Harmon, San Ramon, CA (US)

(73) Assignee: HYDRADX INC., Altadena, CA (US)

(21) Appl. No.: 14/421,618

(22) PCT Filed: Aug. 15, 2013
(86) PCT No.: PCT/US13/55057

§ 371 Date: Feb. 13, 2015

Related U.S. Application Data
(60) Provisional application No. 61/683,637, filed on Aug. 15, 2012.

(51) Int. Cl. A61B 5/00

(52) U.S. Cl. CPC A61B 5/4277; A61B 5/4875; A61B 5/682

(57) ABSTRACT

Detection of one or more fronts of saliva advancing simultaneously in multiple directions may be used to determine salivary secretion rate, as may be indicative of state of euhydration or dehydration. An array of electrical contacts may be arranged between a substrate and a liquid collection element, and used to detect saliva migrating through the liquid collection element. A substrate may include a leading edge defining a notch or recess to receive a sublingual frenulum for positioning a liquid collection element proximate to a sublingual salivary duct, or may include a flexible substrate for positioning a liquid collection element in an oral cavity proximate to at least one parotid or minor salivary duct. Sensing of salivary flow rate may be augmented by measuring concentration of at least one analyte in saliva and/or sensing various environmental factors. Groups of electrical contacts may be arranged proximate to different salivary glands or ducts.

### CROSS-REFERENCE TO RELATED APPLICATION(S)

This application claims priority to U.S. Provisional Patent Application No. 61/683,637 filed on Aug. 15, 2012, and the contents of such application are hereby incorporated by reference herein for all purposes.

### FIELD OF THE INVENTION

The present invention relates to sensing of physiological conditions including hydration state (e.g., euhydration or dehydration) utilizing saliva of mammalian subjects.

### BACKGROUND

Maintaining appropriate hydration level is critical for health and performance of humans and other mammals. Water lost through processes including perspiration and respiration must be replaced. Fluid losses of between 2-3% of body mass detrimentally affect cardiovascular function, thermal dissipation, and exercise performance. Overhydration can also detrimentally affect exercise performance (e.g., due to electrolyte imbalance) and stress a subject's kidneys. Relying upon thirst as a feedback mechanism to trigger demand for fluid intake may not be adequate to maintain an optimal hydration level, since a sensation of thirst sufficient to cause a subject to drink may not be triggered until after the subject is already dehydrated.

One method to assess hydration is to periodically weigh a subject under controlled conditions. For example, over a bout of exercise, a reduction in body weight measured nude before and after exercise will indicate a state of dehydration. While nude body weight changes may be used to assess acute changes in hydration status during a single exercise bout, over longer periods of time, body weight changes may be influenced by many factors other than change in hydration status, such as: food intake, bowel movements, and changes in body composition. As a result, measurement of body weight over a prolonged period is an inaccurate way of assessing whole body hydration status. But even in a single bout of exercise, it may be highly impractical to stop and subject for purposes of measuring nude body weight to assess hydration status.

Other known methods to assess hydration status involve use of testing of urine or blood. For example, urine specific gravity is a common standard among certain physicians. For patients that can be monitored over time, total urine output or urine specific gravity may be used as a metric. Hydration status can also be assessed using a blood sample, since an increase in plasma osmolality can often identify a state of dehydration, but such sensing requires invasive collection of a venous blood sample by a qualified phlebotomist. In numerous settings, use of urine or blood for assessment of hydration status can be highly impractical.

For many reasons, saliva is an ideal choice for development of a rapid, point-of-care diagnostic measurement for dehydration and/or stress. The sample is easily obtained with minimal invasiveness. No blood must be drawn. In many cases, it is difficult for an individual or health care provider to access urine in a patient (especially for the elderly or infants). Also, urine assessment could be indicative of a prior state of dehydration because the urine is maintained in the bladder and does not necessarily reflect a subject's current hydration state. It would be desirable to provide a convenient device and method for sensing hydration status without requiring use of conventional laboratory equipment and specially trained personnel.

Reduced saliva production or hypo-salivation may be caused by certain disease states (e.g., Sjögren's syndrome) or embody a side effect of certain medications. Dry mouth, also called xerostomia, is the medical term for the subjective complaint of dry mouth which is often associated with lack of saliva. Dry mouth can lead to problems with tooth decay, the ability to swallow, and the abilities to taste and digest food. When hypo-salivation is a side effect of one or more medications, such condition may improve with an adjusted dosage or a new prescription.

When saliva production is reduced to an extremely low level (e.g., around 10 microliters per minute or less), it can be difficult to obtain accurate measurements of salivary flow rate using conventional methods. One significant concern is that measured values may be on the same order of magnitude as the range of experimental error. Accordingly, it can be difficult to accurately and reproducibly measure salivary flow rate for subjects with extremely low salivary production. Measurement difficulties at extremely low salivary flow rates also render it very difficult to assess benefits or side effects of medications in relation to salivary function, since a doubling of salivary production rate from, say, 0.3 ml/min to 0.8 ml/min would be beneficial, but may be difficult to detect using conventional methods. It can also be difficult to determine abnormal function of one or more salivary gland relative to other salivary glands of the same mammalian subject, which might be indicative of physiologic abnormalities or disease state (e.g., inflammation, tumors, etc.).

Based on the foregoing, the art continues to seek diagnostic devices and methods for sensing hydration state and/or salivary flow rate of a mammalian subject that are adapted to overcome one or more of the foregoing limitations. It would be desirable to provide non-invasive point of care diagnostic devices and methods capable of sensing salivary flow rate and/or hydration state quickly, accurately, and with a high degree of reproducibility, even for subjects with very low saliva production rates.

### SUMMARY

Certain embodiments according to the present invention involve sampling of saliva within the mouth of a subject proximate to at least one salivary gland duct, to identify a state of euhydration, state of dehydration, or salivary secretion rate. Certain embodiments according to the present invention relate to use of a liquid collection element arranged for placement under the tongue of a subject, with positioning aided by a substrate including a notch or recess arranged to receive at least a portion of the sublingual frenulum of the subject. Certain embodiments include use of a substrate arranged to support a liquid collection element and multiple non-collinearly arranged saliva detection regions (including electrical contacts) arranged to detect a front of saliva absorbing and advancing simultaneously in multiple directions following migration of saliva through the liquid collection element. In certain embodiments, a liquid collection element may be placed into a sublingual area or buccal cavity of a mammalian subject following swabbing of the sublingual area or buccal cavity with an absorptive medium to remove ambient saliva. In certain embodiments, sensing of salivary flow rate may be further augmented by sensing presence and/or concentration of at least one analyte in saliva.

In one aspect, the invention relates to an apparatus for sensing a state of euhydration, state of dehydration, or salivary secretion rate of a mammalian subject, the apparatus comprising: a liquid collection element comprising a hydrophilic, porous medium or fibrous medium; and a substrate including a frontal portion arranged to support the liquid collection element and including a leading edge defining a notch or recess, wherein the notch or recess is arranged to receive at least a portion of a sublingual frenulum of a mammalian subject upon insertion of the frontal portion under a tongue of the mammalian subject. In certain embodiments, a plurality of electrical contacts may be supported by the frontal portion of the substrate, and arranged to detect presence of saliva following migration of saliva through portions of the liquid collection element.

In another aspect, the invention relates to an apparatus for sensing a state of euhydration, state of dehydration, or salivary secretion rate of a mammalian subject, the apparatus comprising: a liquid collection element comprising a hydrophilic, porous medium or fibrous medium arranged to contact oral mucosa of a mammalian subject; a plurality of saliva detection regions comprising a plurality of electrical contacts arranged to detect presence of saliva following migration of saliva through portions of the liquid collection element, wherein the plurality of saliva detection regions includes multiple non-collinearly arranged saliva detection regions arranged to detect a front of saliva advancing simultaneously in multiple directions; and a substrate arranged to support the plurality of electrical contacts and the liquid collection element.

Further aspects of the invention relate to methods utilizing the foregoing apparatuses for sensing a state of euhydration, state of dehydration, or salivary secretion rate of a mammalian subject.

A further aspect of the invention relates to method of sensing a state of euhydration, state of dehydration, or salivary secretion rate of a mammalian subject, the method comprising: swabbing a sublingual area of a mammalian subject with an absorptive medium to remove ambient sublingual saliva; and following said swabbing, inserting under the tongue of the mammalian subject a liquid collection element comprising a hydrophilic, porous medium or fibrous medium supported by a substrate comprising a frontal portion including a notch or recess, wherein said inserting includes positioning the frontal portion to receive at least a portion of a sublingual frenulum of the mammalian subject in the notch or recess.

A further aspect of the invention relates to method of sensing a state of euhydration, state of dehydration, or salivary secretion rate of a mammalian subject, the method comprising: swabbing a portion of an oral cavity between (a) teeth or gums and (b) at least one of a cheek and a lip of a mammalian subject with an absorptive medium to remove ambient saliva proximate to at least one salivary duct; and following said swabbing, inserting into said portion of the oral cavity, proximate to a salivary duct, a liquid collection element comprising a hydrophilic, porous medium or fibrous medium supported by at least a frontal portion of a flexible substrate.

Another aspect of the invention relates to an apparatus for sensing a state of euhydration, state of dehydration, or salivary secretion rate of a mammalian subject, the apparatus comprising: a substrate supporting a plurality of electrical contacts arranged to detect presence of saliva, wherein at least a portion of the substrate is arranged for insertion in the mouth of the mammalian subject; and an accelerometer associated with the substrate and arranged to detect tilt or position of the substrate.

Yet another aspect of the invention relates to an apparatus for sensing a state of euhydration, state of dehydration, or salivary secretion rate of a mammalian subject, the apparatus comprising: a substrate supporting a plurality of electrical contacts disposed in a multi-dimensional array and arranged to detect presence of saliva, wherein at least a portion of the substrate is arranged for insertion in the mouth of the mammalian subject; a processing element arranged to receive signals from the plurality of electrical contacts; and a transmitter or transceiver arranged to receive signals from the processing element and arranged to wirelessly transmit at least one signal indicative of at least one of the following to a terminal or remote communication device: (i) number of electrical contacts triggered within a predetermined time period by saliva in the mouth of the mammalian subject; (ii) rate of triggering of at least some electrical contacts of the plurality of electrical contacts by saliva in the mouth of the mammalian subject; (iii) time within which a predetermined number of electrical contacts of the plurality of electrical contacts are triggered by saliva in the mouth of the mammalian subject; and (iv) a time until changes detected by one or more sensors ceases.

Another aspect of the invention relates to an apparatus for sensing a state of euhydration, state of dehydration, or salivary secretion rate of a mammalian subject, the apparatus comprising: a flexible substrate supporting a plurality of electrical contacts disposed in a multi-dimensional array and arranged to detect presence of saliva; wherein a first portion of the substrate includes a first subset of the plurality of electrical contacts and is arranged for placement in a first location selected from: a sublingual cavity, a left buccal cavity, a right buccal cavity, and a cavity between lower lip and teeth/gums; wherein a second portion of the substrate includes a second subset of the plurality of electrical contacts and is arranged for placement in a second location selected from a sublingual cavity, a left buccal cavity, a right buccal cavity, and a cavity between lower lip and teeth/gums; and wherein the second location differs from the first location; a microprocessor in electrical communication with the plurality of electrical contacts and arranged to receive signals from the plurality of electrical contacts; and a memory arranged to store information derived from the plurality of electrical contacts.

Still another aspect of the invention relates to a method for sensing a state of euhydration, state of dehydration, or salivary secretion rate of a mammalian subject, the method comprising: inserting under the tongue of the mammalian subject a liquid collection element comprising a hydrophilic, porous medium or fibrous medium, a substrate, and a plurality of saliva detection regions including a plurality of electrical contacts arranged to detect presence of saliva following migration of saliva through portions of the liquid collection element, wherein the plurality of saliva detection regions includes multiple non-collinearly arranged saliva detection regions arranged to detect a front of saliva advancing simultaneously in multiple directions; utilizing at least one microprocessor in electrical communication with the plurality of electrical contacts to identify a state of euhydration, state of dehydration, or salivary secretion rate of the mammalian subject based at least in part on one of the following items (i) to (iv): (i) a count of a number of electrical contacts triggered within a predetermined time period by saliva following migration of saliva through portions of the liquid collection element; (ii) a rate of triggering of at least some electrical contacts of the plurality of electrical contacts; (iii) a time within which a predetermined number of electrical contacts of the plurality of electrical contacts are triggered; and generating an output signal indicative of state of euhydration, state of dehydration, or salivary secretion rate of the mammalian subject; and (iv) a time until changes detected by one or more sensors ceases.

Still another aspect of the invention relates to a method for sensing state of euhydration, sensing state of dehydration, sensing salivary secretion rate, or sensing salivary gland function of a mammalian subject, the method comprising: inserting into the mouth of a mammalian subject a flexible substrate supporting a plurality of electrical contacts disposed in a multi-dimensional array and arranged to detect presence of saliva; wherein a first portion of the substrate includes a first subset of the plurality of electrical contacts and is arranged for placement in a first location selected from: a sublingual cavity, a left buccal cavity, a right buccal cavity, and a cavity between lower lip and teeth/gums; wherein a second portion of the substrate includes a second subset of the plurality of electrical contacts and is arranged for placement in a second location selected from a sublingual cavity, a left buccal cavity, a right buccal cavity, and a cavity between lower lip and teeth/gums; and wherein the second location differs from the first location; receiving signals from the plurality of electrical contacts; and performing at least one of the following steps (i) and (ii): (i) mapping salivary secretion rates in the first location and the second location; and (ii) comparing salivary secretion rates in the first location and the second location.

Further aspects of the invention relate to use of the described devices and methods to diagnose a disease state of the mammalian subject, and/or to detect a side effect of an interaction of at least one drug with the mammalian subject. Still further aspects include performing an assay utilizing saliva collected with a liquid collection element to generate a quantitative or qualitative output signal based on concentration of at least one analyte in the saliva.

In a further aspect, any of the foregoing aspects or features and elements as disclosed herein may be combined for additional advantage.

Other aspects, features and embodiments of the invention will be more fully apparent from the ensuing disclosure and appended claims.

### BRIEF DESCRIPTION OF THE DRAWINGS

FIG. 1 is a schematic diagram showing interconnection of various components of a system for sensing a state of euhydration or dehydration of a mammalian subject via saliva of the subject according to one embodiment of the present invention.

FIG. 2A illustrates an open human mouth with upraised tongue to receive a frontal portion of an apparatus for sensing a state of euhydration, state of dehydration, or salivary secretion rate of a mammalian subject, the apparatus including a substrate supporting a liquid collection element, the substrate including a notch or recess arranged to receive at least a portion of the sublingual frenulum for positioning of the liquid collection element under the tongue.

FIG. 2B illustrates the apparatus of FIG. 2A arranged proximate to a vial of buffer solution prior to contacting of a portion of the liquid collection element with the buffer solution.

FIG. 3A is a top assembly view of an apparatus for sensing a state of euhydration, state of dehydration, or salivary secretion rate of a mammalian subject, separately illustrating a substrate with multiple saliva detection regions including an array of electrical contacts arranged to detect presence of saliva, prior to application of a liquid collection element over the array of electrical contacts.

FIG. 3B is a top view of the assembled apparatus of FIG. 3A, following application of a liquid collection element over the array of electrical contacts.

FIG. 3C is a top view of a first alternative arrangement of electrical contacts that may be used with an apparatus according to FIGS. 3A-3B, including contacts arranged along imaginary boundary lines of concentric circles.

FIG. 3D is a top view of a second alternative arrangement of electrical contacts that may be used with an apparatus according to FIGS. 3A-3B, including contacts arranged along imaginary boundary lines of concentric hexagons.

FIGS. 4A-4C illustrate a liquid collection element consistent with the apparatus of FIGS. 3A-3B following absorption of different amounts of liquid.

FIG. 5A is a side cross-sectional schematic view of a portion of an apparatus for sensing a state of euhydration, state of dehydration, or salivary secretion rate of a mammalian subject, the apparatus including a liquid collection element arranged over an array of electrical contacts to detect presence of saliva and supported by a substrate arranged over a convex lower support surface.

FIG. 5B is a side cross-sectional schematic view of a portion of an apparatus for sensing a state of euhydration, state of dehydration, or salivary secretion rate of a mammalian subject, the apparatus including a liquid collection element arranged over an array of electrical contacts to detect presence of saliva and supported by a flexible substrate.

FIG. 6A illustrates separable sampling and monitoring portions of an apparatus for sensing a state of euhydration or dehydration of a mammalian subject via saliva of the subject according to one embodiment of the present invention, with the monitoring portion being connectable via an electrical plug interface to the sampling portion, and the sampling portion including a substrate with notch or recess arranged to receive at least a portion of the sublingual frenulum for positioning of a liquid collection element of the sampling element under the tongue.

FIG. 6B is a photograph of a prototype apparatus for sensing a state of euhydration or dehydration of a mammalian subject including separable monitoring and sampling portions, superimposed over a photograph of a human subject, with a frontal portion of the apparatus including a substrate with notch or recess positioned under the tongue of the subject to receive the sublingual frenulum.

FIG. 7A is a table summarizing results of in vitro testing performed on two different liquid collection element materials to correlate saliva spread diameter (in millimeters) over a 1 minute period with volume of dispensed saliva for a range of saliva volumes from 0.5 to 10 microliters.

FIG. 7B is a line chart plotting diameter of saliva spread (in millimeters) versus volume of dispensed saliva (in microliters) for the test results tabulated in FIG. 7A.

FIG. 8 embodies a table and line chart showing average saliva absorption areas as a function of different pump flow rates at five second intervals over a thirty second period for four liquid collection elements, demonstrating that the faster the saliva flow rate, the greater the area of absorbed saliva over time.

FIGS. 9A-9F embody bar charts showing the time for saliva to spread to specified areas (15, 70, 150, 200, 300, and 400 square millimeters, respectively) at different flow rates (namely, 48, 96, 128, 256, and 466 microliters per minute).

FIG. 10 is a schematic diagram showing interconnection of various components of a system for sensing a state of euhydration, state of dehydration, or salivary secretion rate of a mammalian subject according to another embodiment.

FIG. 11A is a photograph of a portion of a prototype apparatus for sensing a state of euhydration or dehydration of a mammalian subject, the apparatus including a substrate having arranged thereon a multi-dimensional array of electrical contacts and supporting an absorbent liquid collection element, with the substrate including a curved extension supporting a cable arranged to mate with a monitoring/communication interface component including at least one electronic element arranged to receive signals from the electrical contacts and interface with a computing and/or communication device.

FIG. 11B is a photograph of a magnified portion of the prototype apparatus pictured in FIG. 11A, showing the multi-dimensional array of electrical contacts without the liquid collection element, first and second exposed contacts, and a raised registration feature.

FIG. 12A is a photograph of a computer screen including a software user interface representing the multi-dimensional array of electrical contacts and exposed contacts of the prototype apparatus of FIG. 11A in a first state of operation.

FIG. 12B is a photograph of a computer screen including a software user interface representing the multi-dimensional array of electrical contacts and exposed contacts of the prototype apparatus of FIG. 11A in a second, subsequent state of operation.

FIG. 13 is a schematic side cross-sectional view of major salivary glands and selected elements in and around a human mouth, including the parotid gland, the submandibular gland, the sublingual gland, and minor glands along the lower lip.

FIG. 14 is a schematic plan view of a portion (e.g., sampling component) of an apparatus for sensing a state of euhydration, state of dehydration, or salivary secretion rate of a mammalian subject including a flexible substrate with multiple subsets of electrical contacts and supporting a liquid collection element, wherein different portions of the substrate are arranged for placement in different locations of the mouth, such as a sublingual cavity, a left buccal cavity, a right buccal cavity, and a cavity between lower lip and teeth/gums.

### DETAILED DESCRIPTION

The present invention relates in various embodiments to sensing of salivary secretion rate (e.g., as may be measured by collecting of saliva in a liquid collection element inserted proximate to at least one salivary duct of a mammalian subject) as indicative of state of euhydration or dehydration. As noted previously, determination of state of euhydration or dehydration may be further aided by sensing of at least one analyte in saliva.

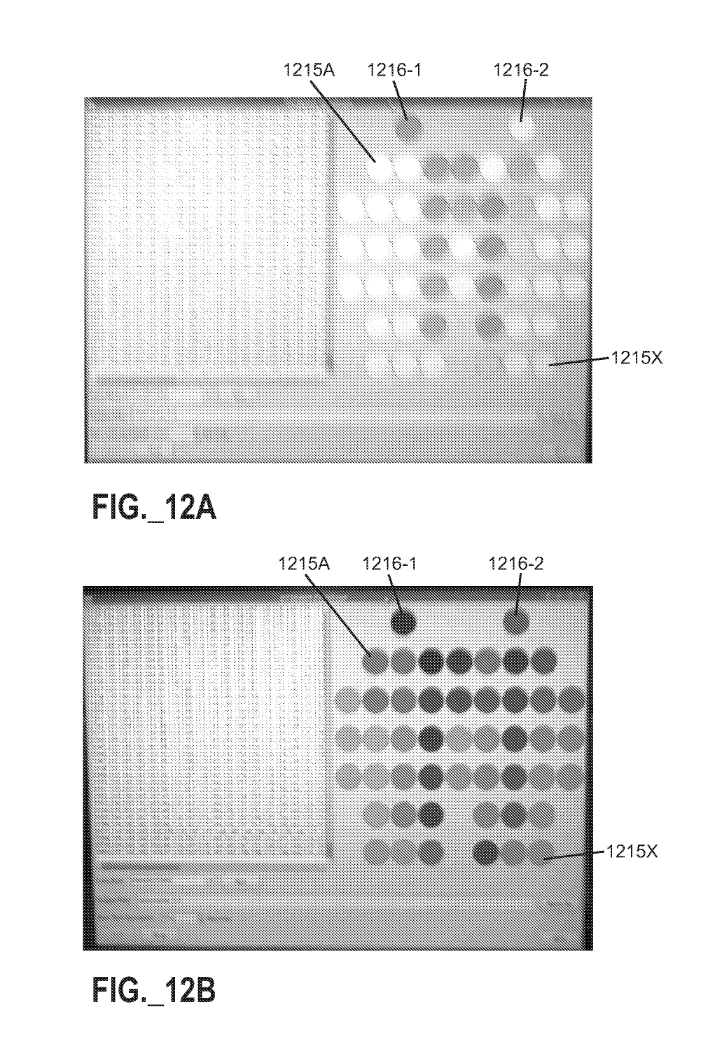

Sources of unstimulated saliva in humans have been determined to be approximately 60% submandibular gland, 7-8% sublingual gland, 25% parotid gland, and 7-8% through other glandular sources. (See Dawes, C., “Salivary flow patterns and the health of hard and soft oral tissues,” JADA 139:18-24, 2008.) FIG. 13 illustrates the parotid gland 1, the submandibular gland 2, the sublingual gland 3, and minor glands 4 along the lower lip. Due to the frequent common exit of the submandibular and sublingual ducts, submandibular saliva is frequently embodying a combined submandibular and sublingual secretion. (See Wu, A. J., et al., “A characterization of major salivary gland flow rates in the presence of medications and systemic diseases,” J. Oral Surg. Oral Med. Oral Pathol. 1993; 76: 301-306.) Wu et al. suggested that the submandibular gland may be more sensitive to physiologic permutations (including conditions and medications leading to salivary hypofunction) than the parotid gland.

Certain aspects of the present invention benefit from recognition by the inventor that (i) placement of a saliva collection element in close proximity to a salivary duct (within the oral cavity of a mammalian subject) enables rapid, accurate, and reproducible saliva collection and saliva flow rate measurement even at very low flow rates, and (ii) multiple non-collinearly arranged saliva detection regions (e.g., including electrical contacts) may be arranged to detect a front of saliva advancing simultaneously in multiple directions following migration of saliva through the liquid collection element.

As opposed to the use of saliva detection regions that are linearly arranged (such as disclosed in U.S. Patent Application Publication No. 2012/0083711 to Goldstein, et al., which is hereby incorporated by reference in its entirety, for all purposes), using multiple non-collinearly arranged saliva detection regions (e.g., including electrical contacts) permits saliva flow rate to be determined rapidly from the multi-directional spread of saliva through a porous or fibrous liquid collection element preferably contained entirely within the oral cavity of a mammalian subject, thereby dispensing with any need for waiting for collected saliva to migrate to or be channeled to a detection region outside the oral cavity or distal to the saliva gland duct, and insuring saliva collection substantially instantaneously with saliva secretion. Various schemes that may be employed using multiple non-collinearly arranged saliva detection regions include (but are not limited to) the following: (i) counting a number of electrical contacts triggered within a predetermined time period by saliva following migration of saliva through portions of the liquid collection element; (ii) determining a rate of triggering of at least some electrical contacts; (iii) determining a time within which a predetermined number of electrical contacts of the plurality of electrical contacts are triggered, and/or (iv) sensing of time to a condition of stasis—i.e., a time to a stop in changes detected by one or more sensors. Although certain embodiments herein refer to the use of electrical contacts arranged to permit closure of circuits by conduction of current though saliva, saliva detection regions may additionally or alternatively use one or more types of sensing elements such as capacitive sensing elements, conductivity sensing elements, optical sensing elements, and the like.

Detection of saliva absorbed by a porous or fibrous liquid collection element as disclosed herein may be used to determine state of euhydration, state of dehydration, state of disease (e.g., where the disease state includes hyposalivation as a symptom thereof), and/or to detect a side effect of interaction of one or more drugs with the mammalian subject. For example, Sjögren's syndrome is a chronic autoimmune disease in which a person's immune system attacks moisture-producing glands, and such disease is characterized in part by hyposalivation. Detection of salivary secretion rate below a predetermined threshold (such as may be optionally validated by multiple runs of substantially the same type, or multiple tests of different types) utilizing devices and/or methods as disclosed herein may be used to diagnose Sjögren's syndrome or detect other disease states (such as may include hyposalivation as a symptom thereof). Similarly, certain drugs are characterized by hyposalivation as a side effect, and devices and/or methods as disclosed herein may be used to detect a side effect of interaction of one or more drugs with a user (e.g., by comparison of an output signal or an apparatus disclosed herein, or information derived from such signal, with at least one reference value or reference value range correlative of the side effect of drug interaction, or comparison to baseline salivary flow information previously established for the same mammalian subject). In certain embodiments, dosage and/or administration of a drug may be adjusted in response to the detection of a side effect (such as hyposalivation) of the drug interaction. In certain embodiments, an apparatus and/or method as disclosed herein may be used to determine suitability or readiness of a patient to undergo a medical or dental procedure that requires or is benefitted by maintenance of a particular saliva secretion rate range or threshold (whether low or high).

Various embodiments of the invention facilitate positioning of a liquid collection element proximate to a sublingual, parotid, or minor salivary duct. In certain embodiments, a liquid collection element including hydrophilic, porous medium or fibrous medium is supported by a substrate that includes a leading edge defining a notch or recess arranged to receive at least a portion of a sublingual frenulum of a mammalian subject, in order to reproducibly position at the frontal portion of a sensing apparatus (including a liquid collection element, preferably in conjunction with multiple non-collinearly arranged saliva detection regions including electrical contacts) under the tongue of a subject proximate to a sublingual salivary duct. In certain embodiments, the hydrophilic, porous medium or fibrous medium is also proteophobic. A frontal portion of such a substrate may be optionally supported by a convex lower surface to conform to the lower surface of the sublingual cavity and facilitate user comfort. Other surface shapes conforming to a sublingual cavity and/or buccal cavity may be employed. In certain embodiments, a liquid collection element including hydrophilic, porous medium or fibrous medium is supported by a flexible substrate (e.g., a thin film substrate), permitting at least a frontal portion of a sensing apparatus (including a liquid collection element, preferably in conjunction with multiple non-collinearly arranged saliva detection regions including electrical contacts) to be positioned in a buccal cavity of a mammalian subject (e.g., between maxillary second molar and cheek) proximate to a parotid salivary duct.

In certain embodiments, the hydrophilic, porous medium or fibrous medium is also proteophobic. In certain embodiments, one or more travel stops or tab portions arranged to be secured by a user by biting (clenching between teeth) may be employed to maintain a sensing apparatus with a liquid collection element in a buccal cavity proximate to a parotid salivary duct.

In certain embodiments, electrical contacts (saliva detection regions) and appurtenant electrical traces may be patterned on a substrate, such as a thin film substrate (e.g., a polymeric thin film), by printing, etching, and/or other conventional circuit patterning techniques. Contacts and/or associated traces may be patterned on one or multiple surfaces of a substrate. Electrically conductive wires or cables may alternatively (or additionally) be used. Multiple positive contacts may be dispersed on a substrate in an array or other pattern, accompanied by one or multiple ground or electrical neutral regions. In certain embodiments, a shared electrical ground region or shared electrically neutral region is arranged between each (e.g., positive) electrical contact of a plurality of electrical contacts. When an advancing front of conductive saliva closes a gap between a positive contact and a ground or neutral region, an electric circuit is closed (or electrical resistance or impedance is reduced). In certain embodiments, electrodes may be provided in complementary pairs, and arranged to form multiple non-collinearly arranged saliva detection regions. In certain embodiments, electrical conductivity of saliva may be enhanced by presence of one or more electrolytes (e.g., sodium chloride) in, on or against a liquid collection element arranged to contact saliva.

In certain embodiments, one or more contacts or electrodes are exposed (e.g., not covered by a liquid collection element) to sense a condition indicative of exposure of a portion of the liquid collection element to oral mucosa and/or salivary duct secretions of a mammalian subject. Such exposed contacts may be used to detect insertion of at least a frontal portion of a sensing device in the mouth of a subject, and automatically establish a starting time (initial time value) and/or automatically start a timer indicative of the start of a sampling period, with an initial time value, elapsed time values, and/or values derived therefrom preferably being stored in a memory associated with a sensing device. In certain embodiments, the exposed contacts may also be used to determine state of the apparatus prior to insertion in the mouth of the mammalian subject, such as by performing a control check for the absence of saliva (e.g., detectable as the absence of closed contacts) prior to initiation of testing/sample collection. One or more time value may be useful in with various saliva flow detection schemes that may be employed using multiple non-collinearly arranged saliva detection regions, such as (i) counting a number of electrical contacts triggered within a predetermined time period by saliva following migration of saliva through portions of the liquid collection element; (ii) determining a rate of triggering of at least some electrical contacts; and (iii) determining a time within which a predetermined number of electrical contacts of the plurality of electrical contacts are triggered. In certain embodiments, a scheme may include sensing of time to a condition of stasis—i.e., a time to a stop in changes detected by one or more sensors.

In certain embodiments, a liquid collection element comprises a porous medium or a fibrous medium arranged to absorb saliva (hydrophilic) and also resistant to adhesion of a protein adlayer (proteophobic). A liquid collection element may be formed of paper, fabric, nitrocellulose, or any other suitable material. In certain embodiments, a liquid collection element comprises a material having directionally uniform liquid wicking characteristics. Thickness, area, porosity, and other parameters of a liquid collection element may be selected to provide a liquid collection element with suitable capacity and absorption characteristics. A liquid collection element may be arranged to cover (and preferably arranged in contact with) multiple saliva detection regions, with the liquid collection element held in place with one or more adhesives or other retaining elements. In certain embodiments, a liquid collection element is retained along peripheral portions thereof via adhesive bonding to a substrate. In certain embodiments, a continuous bead or line of adhesive is provided along peripheral portions of a liquid collection element to prevent saliva from entering into the space between a liquid collection element and multiple saliva detection regions without passage through a face of a liquid collection element. In certain embodiments, at least one peripheral portion of a liquid collection element may be covered with a retaining element (e.g., arranged to compress peripheral portions of a liquid collection against a substrate) that leaves a central portion of the liquid collection element uncovered, with the retaining element resembling a window frame. Such a retaining element may be used to prevent saliva from entering into the space between a liquid collection element and multiple saliva detection regions without passage through a face of a liquid collection element. In certain embodiments, a retaining element is arranged for permanent retention of a liquid collection element; in other embodiment, a retaining element may be arranged to facilitate removal of a liquid collection element, such as by using removable closures and/or hinges.

In certain embodiments, a portion of a liquid collection element may be covered by a removable liquid-impermeable covering element, such as a removable adhesive tape or other covering element. Such a covering element may be arranged to cover a liquid collection element during transit and/or storage to prevent moisture from being absorbed by the liquid collection element, with the covering element subject to being removed prior to use.

In certain embodiments, a liquid collection element may be arranged in or on a sample collection portion of an apparatus for sensing state of euhydration or dehydration, with other components of the apparatus (e.g., timing element, processing element/comparator, input element(s) and/or signaling element(s), among others) being arranged within a monitoring portion. In certain embodiments, a sampling portion may be operatively connected to a monitoring portion via an electrical interface connector (optionally extended with an electrical communication cable), or via wireless communication, thereby permitting the sampling portion to be inserted into the mouth of a user while a monitoring portion is spatially separated therefrom. By providing a sampling portion separate from the monitoring portion, the monitoring portion may be sequentially reused with numerous sampling portions of disposable character. A monitoring portion may be arranged to sense salivary secretion rate alone, or may be arranged to further sense concentration of at least one analyte in saliva (e.g., using an optical reading element and/or any other suitable type of analyzer). A monitoring portion may include any suitable combination of various components such as (but not limited to) a battery, a timer, a processing and/or control element, a comparator, a memory, a display, one or more indicator lights, an audible output element, sensor traces or wires, an electrical interface plug, a sampling portion receiving cavity, an optical reading element (or other sample analyzing element), and a communication element arranged to communicate (whether in wired or wireless fashion) with one or more terminals or other remote communication devices.

In certain embodiments, an apparatus for sensing state of euhydration or dehydration is arranged to generate a user-perceptible output signal indicative of hydration status that is quantitative in character. A user perceptible output signal generated by such an apparatus may be visible, audible, and/or tactile in character. Examples of quantitative signals include salivary secretion rate (e.g., in μl/min or other suitable units, obtainable by dividing volumetric capacity of the liquid collection element over measured time to saturation), number of electrical contacts triggered in a predetermined time period, rate of triggering of at least some electrical contacts, time within which a predetermined number of electrical contacts have been triggered, analyte concentration level (e.g., in μg/ml or other suitable units, obtainable via use of an optical reading element or other analytical element) and the like. In certain embodiments, a sensing scheme may include sensing of time to a condition of stasis—i.e., a time to a stop in changes detected by one or more sensors.

In certain embodiments, a user-perceptible output signal generated by an apparatus for sensing state of euhydration or dehydration with an array of multiple non-collinearly arranged saliva detection regions includes a display arranged to actuate pixels (or groups of pixels) in an array that corresponds to the array of saliva detection regions, such that triggering of individual saliva detection regions (e.g., contacts) results in actuation of corresponding pixels of the display. In this manner, a front of saliva advancing simultaneously in multiple directions following migration of saliva through the liquid collection element may be visualized on a pixel-based display.

In certain embodiments, an apparatus for sensing state of euhydration or dehydration is arranged to generate a user-perceptible output signal indicative of hydration status that is qualitative in character. With respect to salivary secretion, one or more threshold values for salivary secretion rate indicative of euhydration or dehydration may be stored in memory of the apparatus, and sensed values may be compared to the one or more threshold values to provide a simple qualitative assessment as to whether a user is in a state of euhydration or dehydration. A qualitative signal may be selected from two, three, four, or more possible results. For example, in certain embodiments, a qualitative signal may be limited to two possible results of euhydration or dehydration. In other embodiments, a qualitative signal may be selected from three possible results of euhydration, dehydration, and severe dehydration. In other embodiments, a qualitative signal may be selected from four possible results of euhydration, slight dehydration, moderate dehydration, and severe dehydration. If one or more analytes in saliva are analyzed, then output of an optical reading element may be compared to one or more threshold signal level to provide a qualitative output. A processing element and/or comparator may be arranged to compare at least one test value against at least one predetermined threshold value, and/or to compare multiple test values against one another (or one or more values derived therefrom), and responsively trigger a signaling element to generate a user-perceptible qualitative signal indicative of state of euhydration, state of dehydration, or salivary secretion rate based at least in part on such comparison.

In certain embodiments, a hydration state sensing device may provide a quantitative output signal based on results of one salivary test method, and provide a qualitative output signal based on results of one salivary test method. For example, a device may provide a quantitative output signal indicative of salivary secretion rate, and provide a qualitative output signal indicative of analyte concentration (or vice-versa).

One or more user-perceptible output signals representing a quantitative and/or qualitative values may be generated by one or more signaling elements, such as (but not limited to) a LCD display, a LED array, an alphanumeric display, one or more lamps (e.g., LEDs), a sound generating device (e.g., a speaker), and/or a tactile signaling element such as a vibration generator. Multiple signaling elements may be provided. In certain embodiments, a sound generating device may provide an output signal in the form of a synthesized voice.

In certain embodiments, a system for sensing a state of euhydration, state of dehydration, or salivary secretion rate of a mammalian subject may include a sampling component, a monitoring/communication interface component connected via a wired interface to the sampling component, and a remote communication device such as a smartphone, tablet computer, laptop computer, or similar device arranged to communicate wirelessly with the monitoring/communication interface component. Wireless communication may be conducted by Bluetooth Low Energy (BLE) (also known as Bluetooth Smart), Zigbee, or any other suitable wireless communication protocol. At least a portion of a sampling component may be arranged for insertion into a mouth of a mammalian subject, whereas a monitoring/communication interface component may be arranged to be maintained outside the mouth of the mammalian subject. A remote communication device (e.g., smartphone, tablet computer, laptop computer, or similar device) may be loaded with a software application facilitating wireless communication with the monitoring/communication wireless interface component.

In certain embodiments, a monitoring/communication interface component may include a battery, a microprocessor, a wireless transceiver or transmitter, at least one input element arranged to receive a user input, and at least one output element. Examples of input signals that may be communicated via at least one user input element include activation, deactivation, reset, and mode selection. In certain embodiments, at least one output may provide a user-perceptible output signal such an audible signal, a visual signal (e.g., illumination of one or more indicator lights or a display element), and/or a touch-based (e.g., haptic or vibratory) signal. User-perceptible output signals may be used to signal initiation of a test or sampling run, to signal termination of a test or sampling run, to signal a valid or invalid test or sampling run, to signal correct placement or orientation of a sampling component, to signal a salivary secretion rate, and/or to signal a state of dehydration or euhydration.

At least a portion of a monitoring/communication interface component may include an electronic module such as the ConnectBlue OLP425 Bluetooth® Low Energy (BLE) Platform Module (e.g., such as may include BLE chipset, antenna, embedded processor, analog-to-digital converter, battery holder temperature sensor, accelerometers, and/or other sensors) (available from ConnectBlue AB, Malmo, Sweden). Various other types of modules could be used, such as the Bluegiga BLE 112 (Bluegiga Technologies Oy, Espoo, Finland), Blue Radios BR-LE4 0-S2A (Blue Radios, Inc., Englewood, Colo., U.S.A.), Panasonic PAN1720 (Panasonic Electronic Components, Seacaucus, N.J., U.S.A.), or Insight SiP ISP091201 (Insight SiP, Sophia-Antipolis, France). As an alternative to prepackaged modules, BLE chipsets such as the Texas Instruments TI CC2540 or Nordic nRF8001 may be used in conjunction with discrete components to form a monitoring/communication interface component.

In certain embodiments, a monitoring/communication interface component may include one or more sensors arranged to monitor ambient conditions. One or more sensors may be arranged to sense humidity, temperature, pressure, and/or altitude, which may affect a subject's hydration rate. In certain embodiments, output signals from one or more sensors associated with a monitoring/communication interface component may be logged in conjunction with data received or derived from a sampling component, and/or such output signals may be used to validate or assist in determination of a state of dehydration or euhydration of a mammalian subject.

In certain embodiments, a monitoring/communication interface component may include a multiplexing element, which may be embodied in one or more dedicated processors or may include instructions or a routine executed by a single (e.g., central) microprocessor of the monitoring/communication interface component. A multiplexing element may be used to send multiple signals or streams of information on a carrier at the same time in the form of a single, complex signal. In certain embodiments, a multiplexed signal may be transmitted wirelessly from a monitoring/communication interface component to a remote communication device such as a smartphone, tablet computer, laptop computer, or similar device. The remote communication device may be positioned within sufficient range of the monitoring/communication interface component to enable communication of a low power wireless radio frequency signal such as a BLE signal or the like.

In certain embodiments, a monitoring/communication interface component may include at least one timer, which may be embodied in a discrete element or may include instructions or one or more routines executed by a single (e.g., central) microprocessor of the monitoring/communication interface component. In certain embodiments, at least one timer may be used to indicate the start of a sampling period, to indicate elapsed time of a sampling period, to indicate time of closure of one or more electrical contacts, to indicate rate of closure of electrical contacts, and/or to indicate any other time-dependent values. In certain embodiments, a timer may be used to control (e.g., automatically terminate) operation of a wireless transceiver or transmitter associated with a monitoring/communication interface component, in order to conserve battery life by preventing the transceiver or transmitter from being activated more than a specified period (e.g., two to five minutes, or any desired period) after a sampling run has been initiated.

In certain embodiments, a sampling component may include multiple electrical contacts arranged to detect presence of liquid (e.g., saliva) supported by a substrate, wherein at least a portion of the substrate including the contacts may be arranged for placement in the mouth of a mammalian subject. The electrical contacts may be arranged in a multi-dimensional array. At least a portion of the substrate may be flexible. A liquid collection element (e.g., including a hydrophilic, porous medium or fibrous medium that may also be proteophobic in character) may be arranged to cover the electrical contacts, wherein the electrical contacts may be arranged to detect presence of saliva following migration of saliva through portions of the liquid collection element. In certain embodiments, the plurality of electrical contacts may be arranged in physical contact with portions of the liquid collection element. In certain embodiments, a mouth of a mammalian subject may be at least partially cleared of ambient saliva (e.g., by expectoration, swabbing with absorbent media, and/or other means) prior to initiation sample collection, so that presence of saliva detected by the electrical contacts preferably corresponds to saliva secreted contemporaneously with and/or after insertion of the liquid collection device into the subject's mouth. A state of dehydration, state of euhydration, or salivary secretion rate of the mammalian subject based at least in part on one of the following items: count of a number of electrical contacts triggered within a predetermined time period by saliva following migration of saliva through portions of the liquid collection element; a rate of triggering of at least some electrical contacts of the plurality of electrical contacts; a time within which a predetermined number of electrical contacts of the plurality of electrical contacts are triggered; or a time to a stop in changes detected by one or more sensors. At least one exposed electrical contact may be supported by the substrate (e.g., a frontal portion of the substrate) and arranged to detect a condition indicative of placement of the frontal portion into contact with at least one of oral mucosa and salivary duct secretions of the mammalian subject, such as may be useful to automatically initiate operation of a sampling run.

In certain embodiments, a sampling component may include one or more temperature sensors arranged for placement within a mouth of a mammalian subject (e.g., to sense temperature therein) or arranged for placement outside the mouth (e.g., to sense ambient temperature conditions). In certain embodiments, multiple temperature sensors may be arranged in a multi-dimensional array to permit detection of temperature at different locations within a mouth of a mammalian subject. In certain embodiments, multiple temperature sensors may be dispersed among multiple electrical contacts. In certain embodiments, one or more temperature sensors may be uncovered by a liquid collection element. In certain embodiments, one or more temperature sensors may be arranged along a surface of a substrate opposing a surface of the substrate bearing multiple electrical contacts covered by a liquid collection element.

In certain embodiments, a sampling component may include one or more humidity sensors arranged for placement within a mouth of a user (e.g., to sense humidity therein) or arranged for placement outside the mouth (e.g., to sense ambient humidity conditions). In certain embodiments, multiple humidity sensors may be arranged in a multi-dimensional array to permit detection of humidity at different locations within a mouth of a mammalian subject. In certain embodiments, multiple humidity sensors may be dispersed among multiple electrical contacts. In certain embodiments, one or more humidity sensors may be uncovered by a liquid collection element. In certain embodiments, one or more humidity sensors may be arranged along a surface of a substrate opposing a surface of the substrate bearing multiple electrical contacts covered by a liquid collection element.

In certain embodiments, a sampling component may include one or more accelerometers arranged to detect placement or orientation (e.g., tilt) of a substrate when at least a portion of the substrate is placed within a mouth of a mammalian subject. It is believed that salivary secretion rate and/or sample collection may be affected by placement or orientation (e.g., tilt) of a subject's head and/or orientation of at least the portion of a sampling component arranged within the subject's mouth. In certain embodiments, a user-perceptible and/or test administrator-perceptible feedback signal may be generated (e.g., by an output element associated with a monitoring/communication interface component to which the sampling component is connected via a wired interface) based on signals generated by one or more accelerometers based on positioning or orientation of a substrate as affected by positioning or orientation of a subject's head or mouth. In certain embodiments, one or more accelerometers may be associated with (e.g., contacting or otherwise supported by) a substrate, and either arranged for placement within or (more preferably) outside the mouth of a mammalian subject.

The remote communication device may optionally be arranged to communicate (e.g., via a network) with at least one other terminal or other remote device. Wired and/or wireless communication may be employed.

As noted previously, a system for sensing a state of euhydration, state of dehydration, or salivary secretion rate of a mammalian subject may include a sampling component, a monitoring/communication interface component connected via a wired interface to the sampling component, and a remote communication device such as a smartphone, tablet computer, laptop computer, or similar device arranged to communicate wirelessly with the monitoring/communication interface component. In certain embodiments, the remote communication device may provide primary or secondary data processing, primary or secondary data logging/storage, primary or secondary display, and/or primary or secondary condition diagnosis functions. A remote communication device may include a processor (e.g., microprocessor), a display, a memory, at least one input element, and (optionally) a camera or scanning device that may also provide barcode reading functionality. Such a remote communication device may optionally be arranged to communicate (e.g., via a network) with at least one other terminal or other remote device that may have still further data storage and/or data retrieval capability. Utilizing a remote communicate device in wireless communication with a monitoring/communication interface component as outlined above may provide various advantages, including: reduced processing requirements and reduced cost for the monitoring/communication interface component; reduced size/weight of the monitoring/communication interface component; enhanced comfort to the mammalian subject during sampling; enhanced flexibility to the user to store, share, and/or retrieve data; and enhanced access to distributed networks (e.g., the World Wide Web) to facilitate exchange with electronic medical record and/or patient health record systems—which may be aided in particular when a remote communication device includes barcode scanning capability (e.g., for automated merger and/or retrieval of data). Data input via a remote communication device may enable configuration options include steps in a procedure to be performed. A remote communication device may enable retrieval of saliva flow rate reference values. A remote communication device may enable communication with inventory/disposables management and/or support and troubleshooting. A remote communication device may further be used to enter weight of a liquid collection element following completion of a sampling run to compare the entered weight against test results for test validation.

In certain embodiments, a remote communication device may be utilized to capture and store one or more of the following parameters: (i) date/time of sampling run; (ii) patient ID; (iii) patient name; (iv) patient image; (v) sampling data; (vi) error logs; (vii) one or more scanned barcodes; (viii) geolocation via an integrated global positioning chip to identify location of sampling run; (ix) images or video of device placement and/or test administration, and/or (x) graphical representations of test results.

In certain embodiments, one or more control sensors may be provided to provide features such as start timing, timing customization, threshold cutoff on timing, and/or detection of used sampling components.

In certain embodiments, results obtained from a system for sensing a state of euhydration, state of dehydration, or salivary secretion rate of a mammalian subject may be integrated with patient heath records and/or electronic medical records.

In certain embodiments, a substrate of a sampling component may include a first portion arranged for insertion into a mouth of a mammalian subject and a second portion arranged to extend into an ambient environment to interface with a monitoring/communication interface component. In certain embodiments, the first portion includes a leading edge defining a notch or recess, wherein the notch or recess is arranged to receive at least a portion of a sublingual frenulum of a mammalian subject upon insertion of the frontal portion under a tongue of the mammalian subject. In certain embodiments, the second portion may include a flexible and/or curved electrical interface including at least one electrical cable or conductor arranged to permit signals to be conducted to a monitoring/communication interface component. In certain embodiments, part or all of a substrate may be flexible in character. In certain embodiments, a portion of a substrate may be flexible and another portion may be substantially rigid. In certain embodiments, one or more portions of a substrate and/or an electrical interface may be curved in shape and/or include a center of gravity arranged proximate to a chin (or between a chin and neck) of a mammalian subject to facilitate comfort of the subject when at least a portion of the substrate is arranged within a subject's mouth.

In certain embodiments, a liquid collection element may be arranged over a portion of a substrate to cover multiple electrical contacts, and may include a protruding tab or other feature facilitating removal of the liquid collection element (e.g. by peeling) to facilitate subsequent analysis of the liquid collection element (whether by weighing and/or utilization in an assay or another chemical test), to facilitate retention of the liquid collection element in a suitable container, or to permit disposal of the liquid collection element. In certain embodiments, a liquid collection element may include at least one registration feature (e.g., protrusion, hole, or recess) arranged to mate with a corresponding at least one registration feature associated with a substrate (e.g., recess, hole, or protrusion) to facilitate alignment and/or retention of the liquid collection element.

In certain embodiments, instantaneous and/or time-dependent state various contacts and/or sensors associated with a sampling component of a system for sensing a state of euhydration, state of dehydration, or salivary secretion rate may be represented visually using software and a display device. Such software interface and display device may be associated with a remote communication device and/or other terminal or remote device. In certain embodiments, a software user interface may be arranged to color or darken regions to represent closed contacts, and to vary color or vary (e.g., increase) darkness of regions based on increasing elapsed time since initiation of a sampling run. In certain embodiments, state of individual electrical contacts or groups of electrical contacts of a multi-dimensional array of electrical contacts covered with a liquid collection element may be visually represented. In certain embodiments, state of one or more exposed electrical contacts (e.g., for initiating a sampling run) may be visually represented. In certain embodiments, state of individual temperature sensors or groups of temperature sensors of a multi-dimensional array of temperature sensors may be visually represented. In certain embodiments, data obtained from one sampling run may be visually compared to reference data, such as one or more raw or processed (e.g., averaged) sets of data corresponding to previously-captured sampling runs of the same mammalian subject, or one or more sets of raw or processed sets of data corresponding to one or more other mammalian subjects or groups of subjects.

In certain embodiments, an apparatus for sensing a state of euhydration, state of dehydration, or salivary secretion rate of a mammalian subject may include a sampling component that includes a flexible substrate with multiple subsets of electrical contacts, wherein different portions of the substrate are arranged for simultaneous placement in multiple different locations of the mouth, such as two, three, or four of the following locations: a sublingual cavity, a left buccal cavity, a right buccal cavity, and a cavity between lower lip and teeth/gums. Providing multiple subsets of electrical contacts proximate to different salivary glands may permit function of different salivary glands of the same subject to be individually quantified and compared. In certain embodiments, the multiple subsets of electrical contacts may be covered with one or more liquid collection elements. In certain embodiments, at least a frontal portion of the sampling component may include a notch or recess arranged to receive at least a portion of the sublingual frenulum of the subject. In certain embodiments, one or more exposed electrical contacts may not be covered with a liquid collection element, and may be used to automatically signal initiation of a sampling run. In certain embodiments, such as substrate may include one or more additional sensors such as one or more temperature sensors (optionally arranged in a multi-dimensional array), humidity sensors (optionally arranged in a multi-dimensional array), and/or accelerometers as described herein. In certain embodiments, a multi-dimensional array of electrical contacts may overlap or be interspersed with at least one multi-dimensional array of one or more other type(s) of sensors to permit conditions in different portions of a subject's mouth to be separately sampled, monitored, and/or mapped with respect to one or more parameters. In certain embodiments, a heat map of regions of a subject's mouth may be developed using multiple temperature sensors. In certain embodiments, such a heat map may be used to indicate salivary function. In certain embodiments, a sampling component may be used in conjunction with a monitoring/communication interface component connected via a wired interface to the sampling component, and a remote communication device as described herein, and may further be used in conjunction with software (e.g., visualization and comparison software) as described herein.

In certain embodiments, various steps may be performed to prepare a mammalian subject and sensing apparatus for detecting state of euhydration, state of dehydration, or salivary secretion rate and/or perform sensing operations. In certain embodiments, a subject may be prompted to expectorate ambient saliva. The mouth of the subject may be swabbed. Optionally a salivary stimulating agent may be administered. Hardware and/or sensors may be initiated (e.g., including checking for closed/wetted contacts). A sampling component (e.g., including liquid collection element) may be placed in a mouth of the subject and positioned as appropriate for the sampling component (e.g., with portions in one, some, or all of the following locations: sublingual cavity, a left buccal cavity, a right buccal cavity, and a cavity between lower lip and teeth/gums). The subject's head or mouth position or orientation (e.g., tilt) may be sensed with one or more accelerometers. One or more feedback signals may be provided to confirm satisfaction or non-attainment of desired or necessary conditions. Error signals may be handled to display error information such as failure based on control sensors, failed insertion of sensing device, and/or loss of communications. Exposed contacts may be used to initiate data collection and/or initiate operation of a wireless transmitter or transceiver, and initiate operation of a timer used to automatically deactivate the wireless transmitter or transceiver after a conceivable sampling period to conserve battery life of a monitoring/communication interface component. Analysis, display, comparison, and/or trending may be performed utilizing data obtained in a sampling run, optionally in conjunction with data obtained from prior sampling runs. Patient treatment may be performed based on results obtained in a sampling run.

In certain embodiments, a method for sensing state of euhydration, sensing state of dehydration, sensing salivary secretion rate, or sensing salivary gland function of a mammalian subject, the method comprising: inserting into the mouth of a mammalian subject a flexible substrate supporting a plurality of electrical contacts disposed in a multi-dimensional array and arranged to detect presence of saliva; wherein a first portion of the substrate includes a first subset of the plurality of electrical contacts and is arranged for placement in a first location selected from: a sublingual cavity, a left buccal cavity, a right buccal cavity, and a cavity between lower lip and teeth/gums; wherein a second portion of the substrate includes a second subset of the plurality of electrical contacts and is arranged for placement in a second location selected from a sublingual cavity, a left buccal cavity, a right buccal cavity, and a cavity between lower lip and teeth/gums; and wherein the second location differs from the first location; receiving signals from the plurality of electrical contacts; and performing at least one of the following steps (i) and (ii): (i) mapping salivary secretion rates in the first location and the second location; and (ii) comparing salivary secretion rates in the first location and the second location. In certain embodiments, both (i) and (ii) may be performed. In certain embodiments, a substrate includes at least three or at least four portions arranged for placement in different portions of a subject's mouth, and salivary secretion rates are mapped and/or compared in at least three locations or at least four locations.

In certain embodiments, a salivary stimulating agent may be applied to a subject prior to or concurrent with placement of a liquid collection element (or portion thereof) in the subject's mouth, utilizing a device and method for sensing salivary secretion rate. In certain embodiments, a salivary stimulating agent may be coated on or otherwise administered to a user by a liquid collection element. A salivary stimulating agent may be arranged for gustatory and/or olfactory stimulation of saliva production. Examples of gustatory salivary stimulating agents include (but are not limited to) citric acid and sodium chloride. In one embodiment, a saliva stimulant coating may include citric acid (35-45%), sugarless sour candy (54%-64%) and sodium chloride (1%). Mechanical stimulation of saliva production (e.g., a mechanical salivary stimulating element) may also be used. In certain embodiments, a chewable article such as chewing gum is administered to a user prior to or concurrent with placement of a liquid collection element (or portion thereof) in a subject to mouth, in order to stimulate saliva production by chewing.

In certain embodiments, at least one method for sensing a state of euhydration or dehydration as described herein is applied to a user according to a first testing step, one or more output values yielded from the first testing step is stored in a memory, then at least one method for sensing a state of euhydration or dehydration as described herein is applied to the same user according to a second testing step, and one or more output value yielded from the first testing step are compared to one or more stored values. Such steps may be periodically repeated as necessary to assess change in hydration state of the user.

In certain embodiments, at least one of oral fluids, intravenous fluids, electrolytes, medication, and medical treatment may be administered to a user based on one or more output signals generated by hydration state sensing devices and methods as described herein. In certain embodiments, an output signal from an apparatus as described herein may be communicated (e.g., via wired or wireless communication) to an administering element to facilitate automated administration of at least one of oral fluids, intravenous fluids, electrolytes, medication, and medical treatment of fluids without human intervention.

In certain embodiments, operation of one or more sensing methods or processing of results thereof may be affected by one or more signals received from a user input element. In various embodiments, a user input element may be used to store one or more sensed values, to compare one or more currently sensed values to one or more stored values corresponding to previously sensed values, and/or to select a user population of which the user is a member. To the extent that user population status (e.g., age, sex, disease state, medication usage, activity level, or the like) may affect comparison values used to provide qualitative assessments, user input information may be used to adjust or select appropriate comparison values to as a basis for providing a qualitative output signal.

In various embodiments, a liquid collection element may be placed into a portion of an oral cavity between (a) teeth or gums and (b) at least one of a cheek and a lip of a mammalian subject with an absorptive medium to remove ambient saliva proximate to at least one salivary duct. In certain embodiments, the oral cavity portion may include a buccal cavity, and the at least one salivary duct may include at least one parotid duct. In certain embodiments, the oral cavity portion may include a cavity between (a) lower teeth or gums and (b) a lower lip of the mammalian subject, and the at least one salivary duct may include at least one minor salivary duct. In certain embodiments, the oral cavity portion may include a sublingual cavity, and the at least one salivary duct may include a sublingual salivary duct. Such swabbing may be useful to remove ambient saliva, thereby reducing a potential source of variability in determining saliva flow rate. In certain embodiments, an apparatus for sensing a state of euhydration, state of dehydration, or salivary secretion rate of a mammalian subject may be packaged together with at least one swab (to perform the foregoing swabbing function) and usage instructions in a kit. As an alternative to or in addition to (e.g., preceding) swabbing, a subject may be prompted to expectorate and/or swallow in order to remove ambient saliva from at least a portion of the oral cavity.

In certain embodiments, a liquid collection element, sampling portion, and/or an entire device (which may or may not include saliva detection regions and/or electrodes) as described herein may be weighed following collection of a saliva sample to determine an amount of collected saliva. For example, a liquid collection element, sampling portion, and/or an entire device may have a predetermined weight or may be weighed before (or contemporaneously with) administration to a user, and following placement of a liquid collection element and/or sampling portion into the mouth of a user (e.g., to position the liquid collection element sublingually or in a buccal cavity) for a specified time period, the liquid collection element, sampling portion, and/or entire device may be weighed (e.g., using a high precision digital scale), whereby the amount of weight gained represents absorbed saliva. Such determination of salivary flow rate may be used in conjunction with any of various devices as disclosed herein as an alternative to, or in addition to, one or more salivary flow rate measurement techniques described herein. In certain embodiments, saliva flow rate may be determined utilizing the difference in weight of the at least a portion of the salivary diagnostic device and utilizing a difference in time from (i) a time initiated by triggering of at least one saliva detection region to (ii) a time indicative of an end of saliva collection by the liquid collection element.

In certain embodiments, a liquid collection element utilized for sensing salivary secretion rate may embody, or may be provided in fluid communication with, an immunochromatographic lateral flow test strip including at least one indicator or test region arranged to interact with at least one analyte in saliva obtained from the mammalian subject, so that a salivary analyte concentration in sensing method may be performed following the sensing of salivary secretion rate utilizing the same saliva sample. A conjugate pad (e.g., including monoclonal antibodies conjugated to gold nanoparticles) employed by an immunochromatographic assay for sensing salivary analyte concentration may be arranged between a liquid collection element and an immunochromatographic test strip. One or more analytes in saliva may be correlated to hydration level. For example, research recently performed by Dr. Neil P. Walsh of the University of Wales (Bangor) School of Sport, Health and Exercise Sciences (as subsidized by the assignee of the present application, and published as Appl. Physiol. Nutr. Metab. 37:1-10 (June 2012)) has demonstrated that dehydration is accompanied by a reduction in salivary secretion rate, as well as increases in certain analyte contained in saliva, such as IgA and albumin. It has also been theorized that dehydration may be accompanied by an increase in salivary aldosterone concentration may be correlative of dehydration.

In certain embodiments, a liquid collection element and an immunochromatographic test strip may be arranged in or on a common substrate (with at least portions thereof optionally contained in a housing). Alternatively, an electrochemical immunoassay may be utilized. In other embodiments, a liquid collection element is associated with a first substrate, and an immunochromatographic test strip is associated with a second substrate or housing. Following completion of a salivary secretion rate sensing method, a liquid collection pad may be treated with a buffer solution (e.g., by dipping a liquid collection element in a container (e.g., vial) of buffer solution), and a buffered saliva composition may be supplied to an immunochromatographic test device. One potential benefit of such buffering is to mitigate variability in saliva samples (e.g., viscosity, tonicity, ionic strength, and/or pH) that may otherwise reduce reliability of an immunochromatographic assay.

In certain embodiments, an immunochromatographic lateral flow test strip may include a uniform layer of monoclonal antibodies adapted to bind a selected analyte in saliva. Examples of suitable analytes include (but are not limited to) IgA (e.g., secretory IgA, or SIgA), salivary albumin, secretory component, and salivary aldosterone, and while SIgA is mentioned hereafter, it is to be understood that any suitable analyte may be used. An immunochromatographic lateral flow test strip may be designed to bind a calibrated quantity of SIgA (e.g., using anti-SIgA) per millimeter of strip length. As saliva enters the strip, the SIgA may be colorized by passing through a conjugate pad containing anti-SIgA conjugated to nano-gold particles which binds to the SIgA, imparting a color (e.g., pink) to the analyte. When all of the colored SIgA is bound to the strip, the remaining length of the strip remains white. Marks may be delineated by a cover optionally containing multiple holes that reveal the strip at various points along its length, with each pointing represents a total amount of SIgA per ml of saliva. Calibration marks may be arranged proximate to openings in the cover to permit quantitative assessment of analyte concentration based upon presence (or absence) of color in an adjacent window.

In certain embodiments, an immunochromatographic lateral flow test strip may include a series of antibody (e.g., anti-SIgA) bands arranged as stripes aligned perpendicular to a long dimension of the strip. Each stripe line has a specific (though not necessarily the same) analyte absorptive capacity. Thus, substantially the same method as articulated in the preceding paragraph can be accomplished using a series of striped bands rather than a uniform layer of antibodies for the selected analyte over the entire strip.

In certain embodiments, an immunochromatographic lateral flow test strip and cover may be arranged to provide a positive control region (e.g., corresponding to a window defined in a cover over the strip) disposed downstream of a conjugate pad but upstream of one or more indicator windows providing quantitative or qualitative indication of hydration state. Colorization of such a positive control region (e.g., with pink color) will indicate that a liquid sample is present within the lateral flow test strip, that the conjugate pad is functional, and that the sample contains the analyte (or analytes) of interest. Presence of a colored signal in the positive control region may increase confidence in quantitative or qualitative signals generated in downstream test windows.

In certain embodiments, one or more immunochromatographic lateral flow test strips or portions thereof within the same apparatus may include different monoclonal antibodies arranged to bind different analytes. In certain embodiments, an immunochromatographic lateral flow test strip may include a first group of monoclonal antibodies arranged to interact with a first analyte selected from IgA, albumin, secretory component, and aldosterone, and a second group of different monoclonal antibodies arranged to interact with a second analyte selected from IgA, albumin, secretory component, and aldosterone, wherein the second analyte differs from the first analyte. Different antibodies arranged to interact with different analytes may be provided in one or more of same test or indicator regions located along a single immunochromatographic lateral flow test strip, may be provided in different test or indicator regions located along parallel flow paths in a single immunochromatographic lateral flow test strip, or may be provided in different test or indicator regions located in multiple immunochromatographic lateral flow test strips arranged in parallel within a single device or system. Different analytes may be colored differently (e.g., blue and yellow) via one or more conjugate pads. Signals based upon a combination of different analytes bound in the same region may be combined (e.g., blue and yellow combined to make a green color). lmmunochromatographic lateral flow test strips arranged to interact with multiple analytes may be arranged to perform different assay formats, such as a competitive binding assay format or a sandwich assay format.

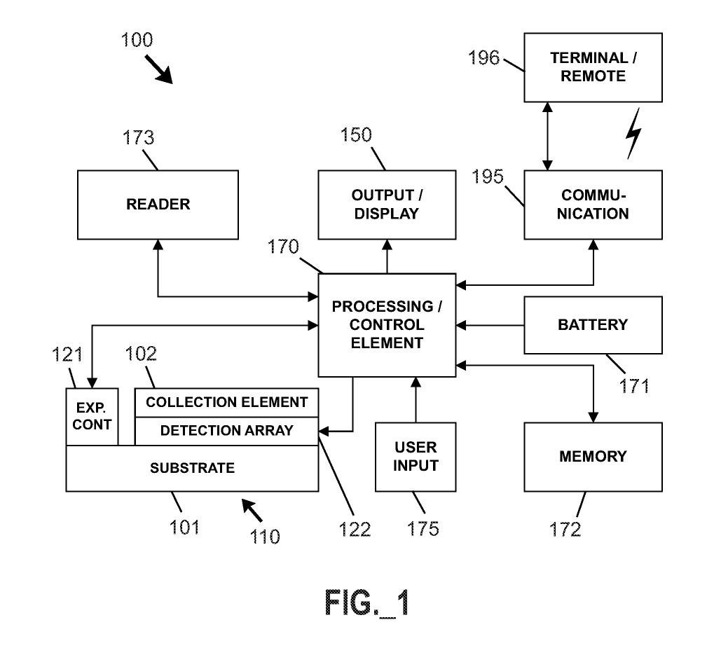

Referring to the figures, a schematic diagram showing interconnection of various components of a system 100 for sensing a state of euhydration or dehydration of a subject via saliva of the subject is illustrated in FIG. 1. A sampling portion 110 includes a substrate 101 supporting an array of multiple non-collinearly arranged saliva detection regions (including electrical contacts) 122, a liquid collection element 102 covering the saliva detection regions 102, and at least one exposed electrical contact 121 not covered by the liquid collection element 102. The saliva detection regions 122 are arranged to detect a front of saliva advancing simultaneously in multiple directions following migration of saliva through the liquid collection element 102. A sampling (frontal) portion 110 is insertable into the mouth of a mammalian subject (for example, into a sublingual area or buccal cavity). The at least one exposed electrical contact 121 may be used to automatically determine when the liquid collection element is placed in contact with oral mucosa (or saliva) of the mammalian subject, and may responsively start a timing element (e.g., a timer). The exposed contact(s) 121 and saliva detection regions 122 are arranged in electrical communication with a processing/control element 170 having an associated battery 171 and a memory 172. In certain embodiments, the processing/control element 170 comprises a microprocessor arranged to execute a predefined machine-readable instruction set. The processing/control element 170 may provide timing functionality, or a separate timer (not shown) may be used. A signaling or output element 150, which may be embodied in a display, is arranged in electrical communication with the processing/control element 170. An optional reading element 173 such as an optical reader may be arranged in communication with the processing/control element 170. The processing/control element 170 may receive user input signals from a user input element 175. A communication element 195, such as may be embodied in a wired interface (e.g., a plug or socket) and/or wireless interface (e.g., a transmitter or transceiver), may be arranged to permit communication and signals between the processing/control element 170 and at least one terminal or remote communication device 196. In this manner, signals indicative of state of euhydration or dehydration of a user may be monitored and logged remotely (e.g., at a nurse's station or a sport manager's communication device), and actions may be taken responsive to such signals. In certain embodiments, a terminal or remote communication device 196 arranged to receive signals indicative of state of euhydration or dehydration may be worn by a participant and/or another person (e.g., a coach or support personnel) while a participant is participating in an athletic event.

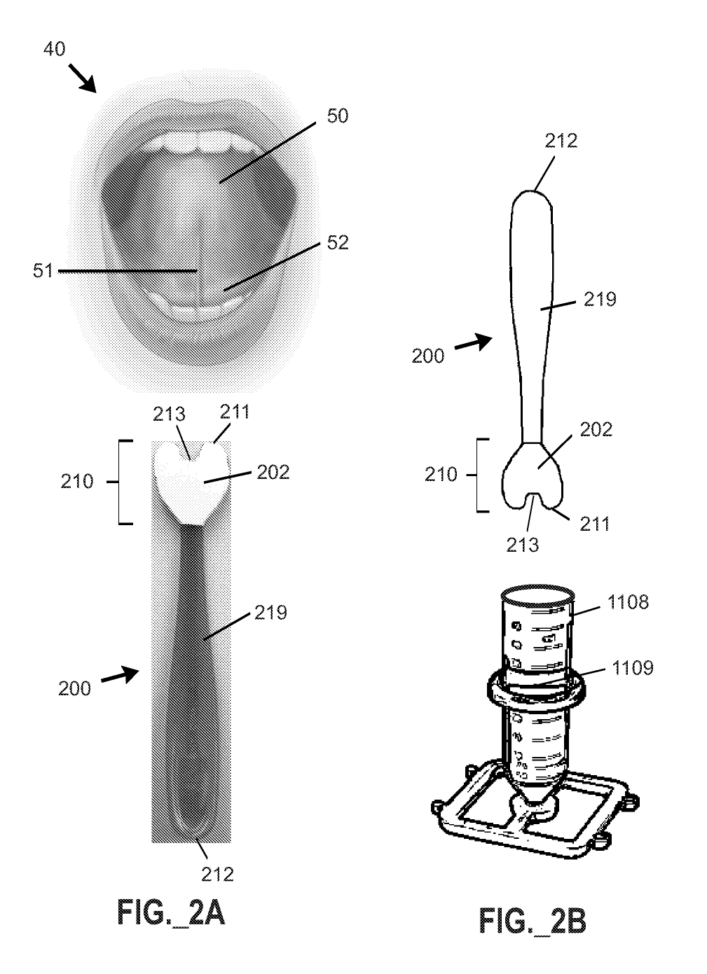

FIG. 2A illustrates an open human mouth 40 with upraised tongue 50 to receive a frontal portion 210 of an apparatus 200 for sensing a state of euhydration, state of dehydration, or salivary secretion rate of a mammalian subject, the apparatus 200 including a substrate 219 supporting a liquid collection element 202 along the frontal portion 210. The substrate 219 includes a notch or recess 213 proximate to an insertion end 211 or the apparatus 200, with the notch or recess 213 arranged to receive at least a portion of the sublingual frenulum 51 for positioning of the liquid collection element 202 and the frontal portion 210 under the tongue 50 in the sublingual cavity 52. Presence of the notch or recess 213 to receive the frenulum 51 aids in retaining the liquid collection element 202 proximate to a sublingual salivary duct. A distal end 212 of the substrate 219 is arranged to permit a user to hold the apparatus 200, wherein the distal end 212 remains outside the mouth 40 of the mammalian subject.

FIG. 2B illustrates the apparatus 200 of FIG. 2A arranged proximate to a vial 1108 of buffer solution 1109 prior to contacting of the liquid collection element 202 and frontal portion 210 of the apparatus 200 with the buffer solution 1109. As noted previously, a liquid collection element may be treated with a buffer solution to yield a buffered saliva composition, and a buffered saliva composition may be supplied to an immunochromatographic test device (not shown) in order to perform an immunochromatographic assay, electrochemical immunoassay, or other chemical test, to identify presence and/or concentration of one or more analytes in saliva.

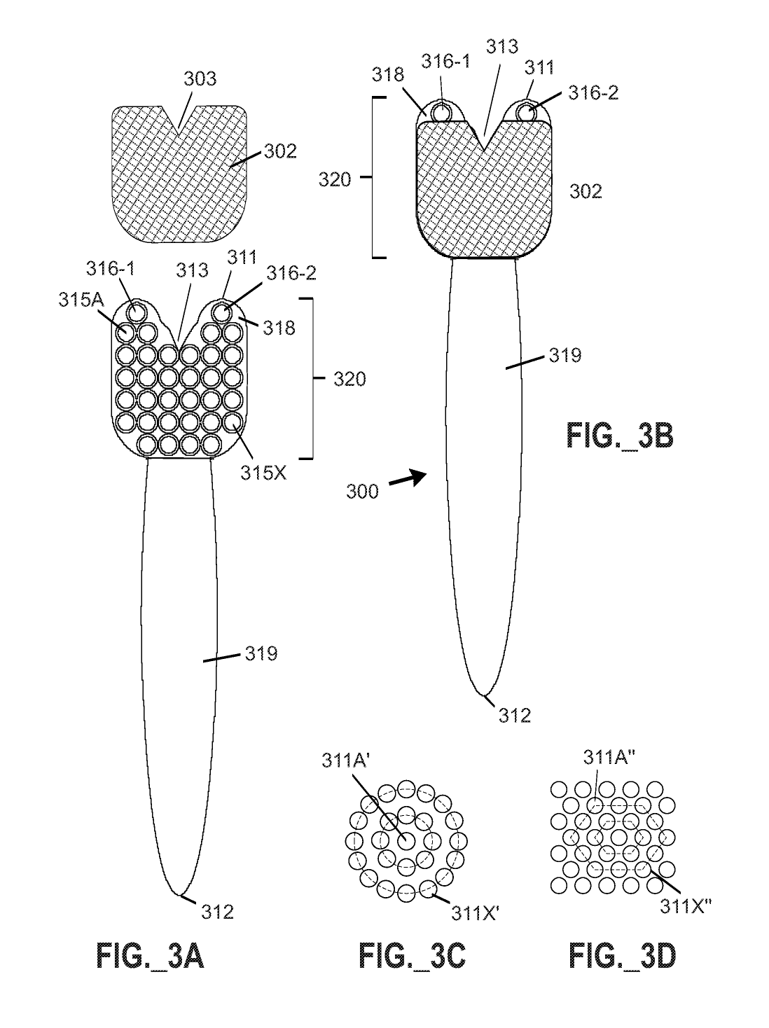

FIG. 3A illustrates a top assembly view, and FIG. 3B illustrates a top assembled view, of an apparatus 300 for sensing a state of euhydration, state of dehydration, or salivary secretion rate of a mammalian subject. The apparatus 300 includes a substrate 319 with a frontal portion 320, an insertion end 311, a distal end 312, and a notch or recess 313 defined in the substrate 319 proximate to the insertion end 311, with the notch or recess 313 adapted to receive a sublingual frenulum upon insertion of the frontal portion 320 into the sublingual cavity of a mammalian user. Proximate to the insertion end 311, exposed electrical contacts 316-1, 316-2 are arranged along an upper surface of the substrate 319, together with a shared ground region or shared electrically neutral region 318. The frontal portion 320 of the substrate 319 further includes multiple non-collinearly arranged saliva detection regions 315A-315X (including electrical contacts) arranged to detect a front of saliva advancing simultaneously in multiple directions. (Although the saliva detection regions are identified with alphanumeric suffixes “A” through “X”, the term “X” represents a variable and is not limited to any specific quantity or number of detection regions). The saliva detection regions 315A-315X are embodied in an array of electrical contacts dispersed among the shared ground region or shared electrically neutral region 318. A small gap is provided between each electrical contact 315A-315X and the shared ground or neutral region 318, whereby presence of conductive saliva across a gap closes a conductive path between a contact 315A-315X and the ground or neutral region 318. Electrical traces or other conductors such as wires (not shown) may be arranged between the saliva detection regions 315A-315X and a processor (not shown) associated with the substrate 319 or another body structure. Although the apparatus 300 is illustrated with round saliva detection regions 315A-315X arranged in an array of orthogonal rows and columns, it is to be appreciated that saliva detection regions may be provided in any suitable number, shape, and configuration. In certain embodiments, multiple non-collinearly arranged saliva detection regions include multiple electrical contacts arrangeable along imaginary boundary lines of multiple concentric geometric shapes traceable over the substrate. For example, FIG. 3C illustrates electrical contacts 311A′-311X′ arranged along imaginary boundary lines of concentric circles, and FIG. 3D illustrates electrical contacts 311A″-311X″ arranged along imaginary boundary lines of concentric hexagons. Moreover, any of various suitable types of sensors may be utilized instead of or in addition to such electrodes.

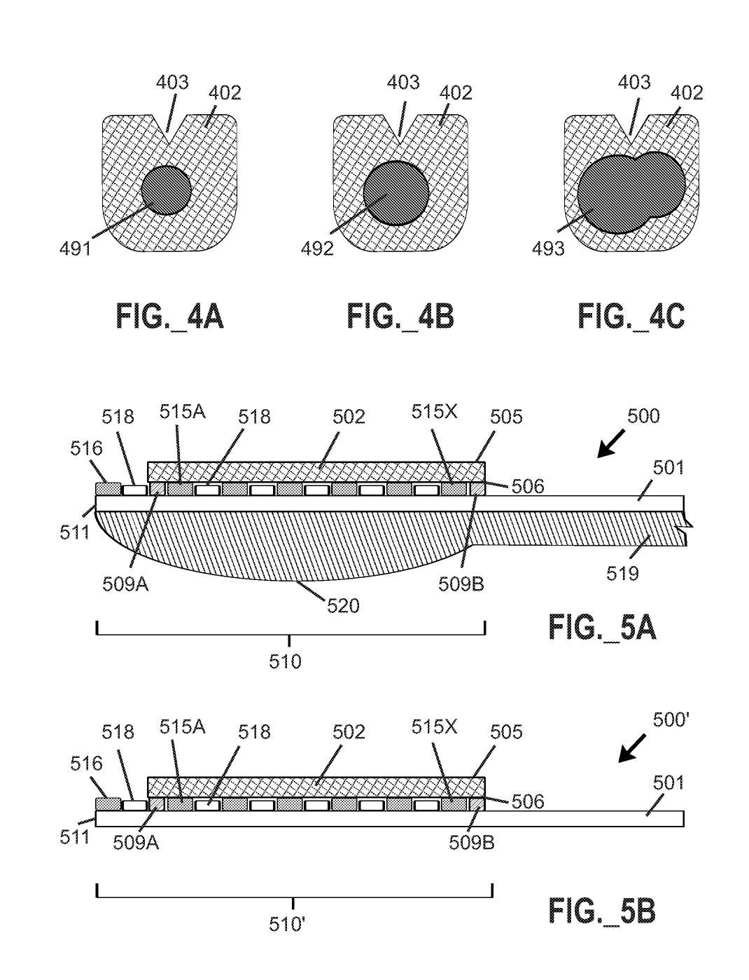

FIGS. 4A-4C illustrate a liquid collection element 402 with notch or recess 403 consistent with the liquid collection element of the apparatus of FIGS. 3A-3B, following absorption of different amounts of liquid (e.g., saliva). FIG. 4A illustrates a saturation zone 491 covering a first portion of the liquid collection element 402; FIG. 4B illustrates a saturation zone 492 covering a second portion (larger than the first portion) of the liquid collection element 402; and FIG. 4C illustrates a saturation zone 493 covering a third portion (larger than the second portion) of the liquid collection element 402. Such saturation zones 491, 492, 493 may be obtained in sequence based on duration of exposure of a liquid collection element 402 to oral mucosa (e.g., in a sublingual cavity) of a user, and together how front of saliva can advance simultaneously in multiple directions upon absorption into the liquid collection element 402.

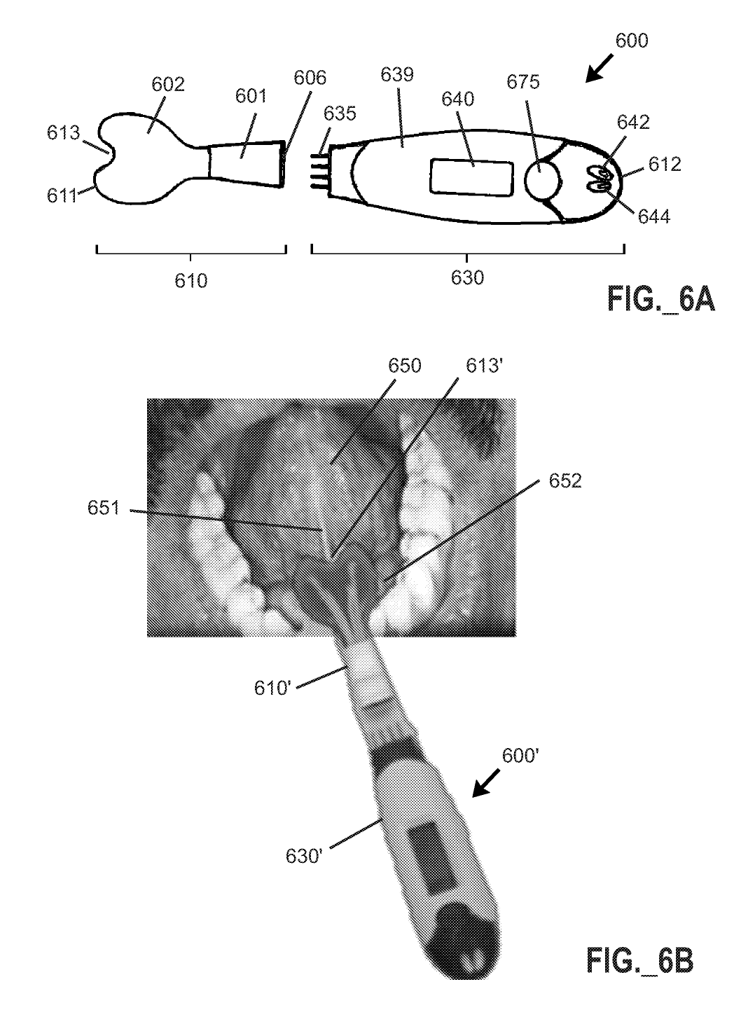

FIG. 5A is a side cross-sectional schematic view of a portion of an apparatus 500 for sensing a state of euhydration, state of dehydration, or salivary secretion rate of a mammalian subject. A frontal portion 510 of the apparatus 500 includes a liquid collection element 502 (including an outer surface 505 and an inner surface 506) arranged over an array of saliva detection regions 515A-515X (including electrical contacts) dispersed along a shared ground or neutral region 518 to detect presence of saliva, and supported by a substrate 501 arranged over a convex lower support surface 520 of a support element 519. The lower support surface 520 is preferably sized and shaped to conform to a lower surface of a sublingual cavity; although a convex shape is illustrated, the lower support surface 520 may be provided in any suitable shape or conformation. Adhesive regions 509A, 509B are provided along peripheral portions of the liquid collection element 502 to adhere the liquid collection element 502 to the substrate 501 and to prevent saliva from entering into a space between the liquid collection element 502 and the saliva detection regions 515A-515X without passage through upper and lower faces 505, 506 of the liquid collection element 502. At least one exposed electrical contact 516 and a portion of the shared ground or neutral region 518 are arranged over the substrate 501 proximate to an insertion end 511 of the substrate 501 (and not covered by the liquid collection element 502) in order to automatically determine when the a frontal portion 510 is inserted into the mouth of a mammalian subject and in contact with oral mucosa or saliva.

Although FIG. 5A illustrates the liquid collection element 502 as being flat in character, in certain embodiments a liquid collection element may be arranged in one or more non-planar (e.g., concave and/or convex) configurations. In certain embodiments, a top surface of a support element and/or substrate is convex, and a liquid collection element is arranged to conform to such a top surface in a non-planar conformation.

FIG. 5B is a side cross-sectional schematic view of a portion of another apparatus 500′ for sensing a state of euhydration, state of dehydration, or salivary secretion rate of a mammalian subject similar to the apparatus 500 of FIG. 5A, but lacking a support element 519. A frontal portion 510′ of the apparatus 500′ includes a flexible substrate 501 arranged to support at least one exposed contact 516 (proximate to insertion end), saliva detection regions 515A-515X, a shared ground or neutral region 518, adhesive regions 509A-509B, and a liquid collection element 502 (including outer and inner faces 505, 506) arranged over the saliva detection regions 515A-515X. Due to the flexible character of at least the frontal portion 510′ of the apparatus 500′, the frontal portion 510′ may be suitable insertion into a buccal cavity of a mammalian subject (e.g., between maxillary second molar and cheek) proximate to a parotid salivary duct to facilitate transfer of saliva from such the parotid salivary duct to the liquid collection element 502.

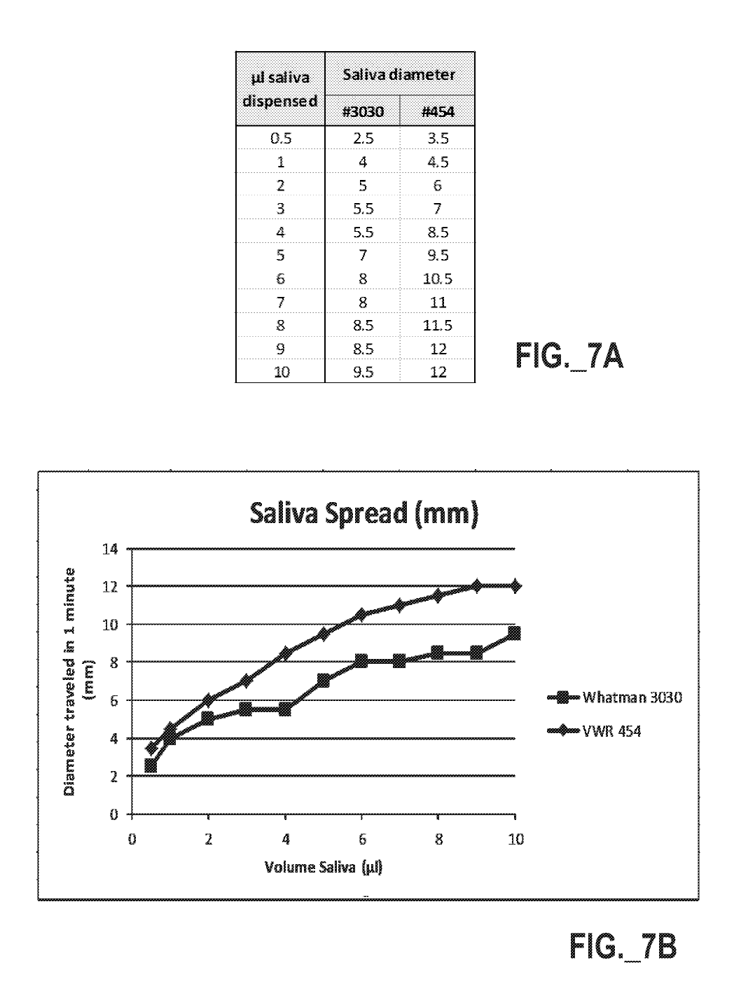

In certain embodiments, separable sampling and monitoring portions of a hydration state sensing apparatus may be connected via an electrical cable and/or other wired or wireless interface. Referring to FIG. 6A, an apparatus 600 for sensing a state of euhydration, state of dehydration, or salivary secretion rate of a mammalian subject includes a sampling portion 610 and a monitoring portion 630 connected via male electrical plug 635 and a corresponding female electrical socket 606. The sampling portion 610 is intended for disposable one-time use, whereas the monitoring portion 630 is intended to be re-used with different sampling portions 610. A processing/control element, battery, and timing element (not shown) associated with the monitoring portion 630 may be provided in electrical communication with electrodes and/or sensors (not shown) of the sampling portion 610 via the interfacial plug 635 and socket 606. The sampling portion 610 includes a liquid collection element 602 and an insertion end 611 defining a notch or recess 613 arranged to receive a sublingual frenulum upon insertion of the insertion end into a sublingual cavity of a mammalian subject. A substrate or body 610 supports the liquid collection element 602. The interface socket 606 is arranged proximate to a distal end of the sampling portion 610. The monitoring portion 630 includes a user input element (e.g., button) 675, a signaling element 640 which may comprise a display, and additional signaling elements 642-644 which may comprise visual signaling elements (e.g., alphanumeric and/or pixel-based display) or auditory signaling elements, and which may be positioned near a distal end 612. In certain embodiments, a visible signal is provided by a display element (e.g., display 640) may be arranged to display a “+” symbol if the user's salivary flow rate is consistent with a state of dehydration, and a “−” symbol if the user's salivary flow rate is consistent with a state of euhydration. In certain embodiments, the display element 640 may be arranged to actuate pixels (or groups of pixels) in an array that corresponds to the array of saliva detection regions, such that triggering of individual saliva detection regions (e.g., contacts) results in actuation of corresponding pixels of the display element 640. In certain embodiments, the monitoring portion 630 may include at least one optical reader or other analytical device (not shown), such as may be used to aid in performing or obtaining results from an immunochromatographic assay (e.g., if the liquid collection element 602 includes or is arranged in fluid communication with a immunochromatographic test strip).

FIG. 6B is a photograph of an prototype apparatus 600′ for sensing a state of euhydration or dehydration of a mammalian subject including separable monitoring and sampling portions 630′, 610′, superimposed over a photograph of a human subject, with a frontal portion of the apparatus including a substrate with notch or recess 613′ positioned under the tongue 650 of the subject to receive the sublingual frenulum 651.

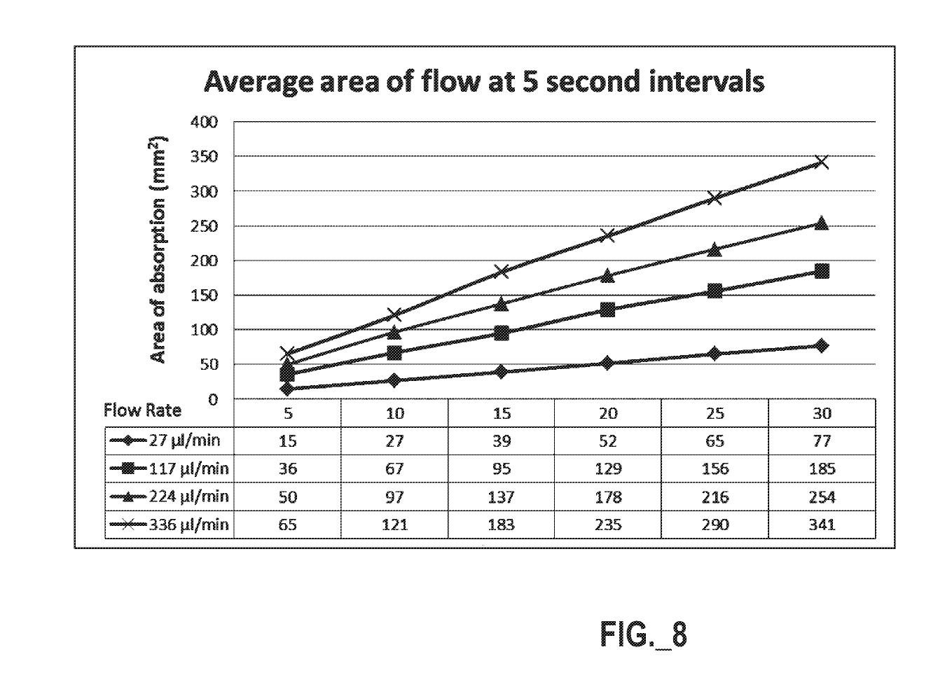

FIG. 7A is a table summarizing results of in vitro testing performed on two different liquid collection element materials (i.e., VWR LabShop Grade 454 filter paper, 0.18 mm thickness (VWR LabShop, Batavia, Ill.) and Whatman™ 3030 3MM Chr cellulose blotting paper, 0.34 mm thickness (GE Healthcare)) to correlate saliva spread diameter (in millimeters) over a 1 minute period with volume of dispensed saliva for a range of saliva volumes from 0.5 to 10 microliters. As shown in FIG. 7A, saliva spread diameter increased with increasing volume of dispensed saliva (dispensed as a bolus from a pump), with the thicker collection element producing smaller saliva spread diameter. FIG. 7B is a line chart plotting diameter of saliva spread (in millimeters) versus volume of dispensed saliva (in microliters) for the test results tabulated in FIG. 7A. FIGS. 7A-7B demonstrate that saliva spread area (absorption area) may be correlated to volume of saliva dispensed to and absorbed by a liquid collection element.

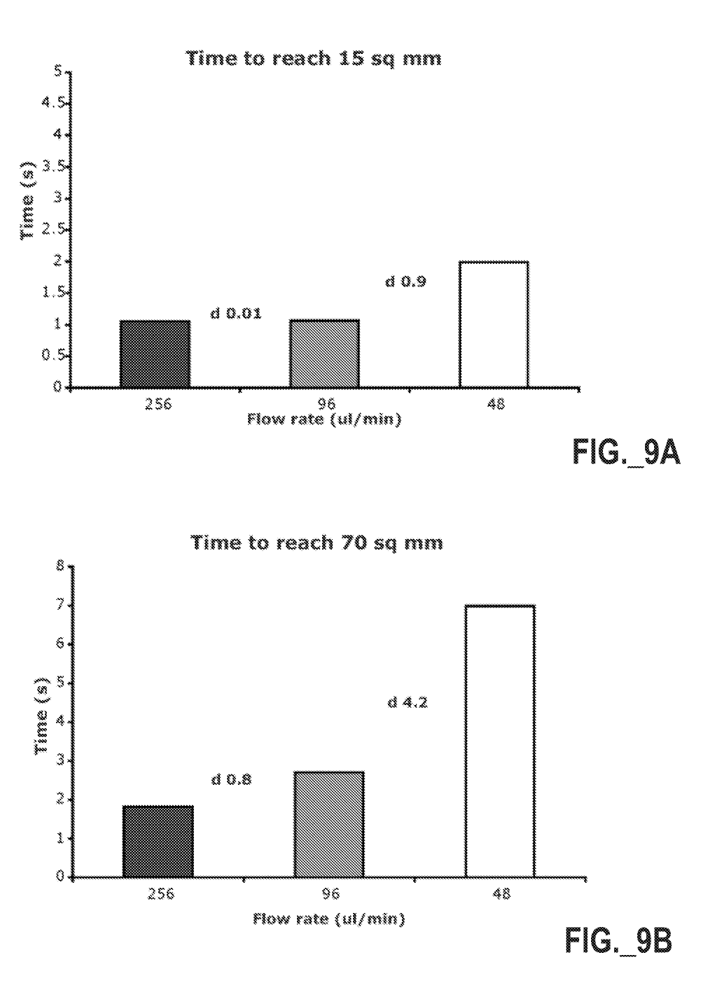

FIG. 8 embodies a table and line chart showing average saliva absorption areas (in square millimeters) as a function of four different pump flow rates (27, 117, 224, and 336 μl/min, respectively) at five second intervals over a thirty second period for four liquid collection elements. Both the table and line chart of FIG. 8 show that the faster the saliva flow rate, the greater the area of absorbed saliva over time. FIG. 8 therefore demonstrates that saliva absorption rate is correlated to saliva flow rate.

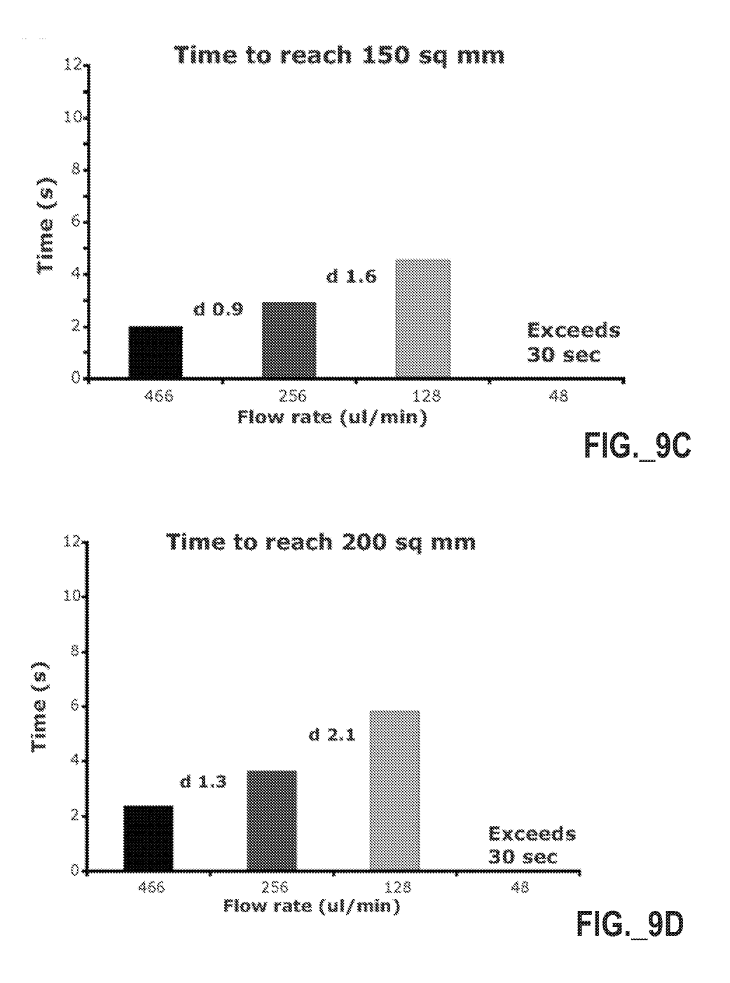

FIGS. 9A-9F embody bar charts showing the time for saliva to spread to specified areas (15, 70, 150, 200, 300, and 400 square millimeters, respectively) at different flow rates (namely, 48, 96, 128, 256, and 466 microliters per minute). FIGS. 9A-9F show that different flow rates are distinguishable at different absorption times.

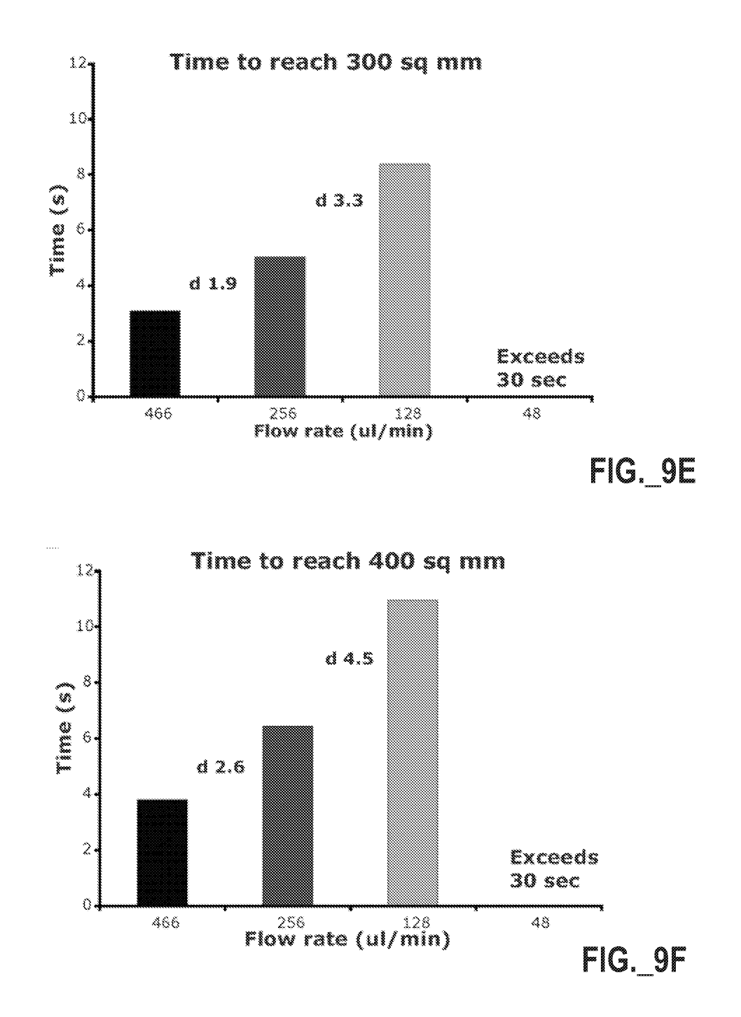

FIG. 10 is a schematic diagram showing interconnection of various components of a system 1000 for sensing a state of euhydration, state of dehydration, or salivary secretion rate of a mammalian subject, including a sampling component 1007, a monitoring/communication interface component 1030, and a remote communication device 1086. The sampling component 1007 in combination with the monitoring/communication interface component 1030 may embody a monitoring subsystem 1008. The sampling component 1007 may include a substrate supporting electrical contacts 1015 (e.g., for saliva detection), temperature sensor(s) 1024, exposed contact(s) 1016, humidity sensor(s) 1023, and acceleration sensor(s) 1025. A liquid collection element 1002 may be arranged to cover the electrical contacts 1015 and possibly cover some or all of the temperature sensor(s) 1024. At least a portion the substrate 1001 may be flexible in character. Components of the substrate 1001 are arranged in electrical communication with the monitoring/communication interface component 1030 by way of one or more interface cables or conductors 1035, which may be flexible in character. The monitoring/communication interface component 1030 may include a battery 1071, a microprocessor 1070, a multiplexer element 1094, a timer element 1072, a wireless transceiver or transmitter 1094, at least one input element 1075 arranged to receive a user input, and at least one output element 1040. The wireless transceiver or transmitter 1094 is preferably arranged to communication wirelessly with a remote communication device 1086 (e.g., smartphone, tablet computer, laptop computer, or similar device) via a wireless communication protocol such as Bluetooth Low Energy (BLE) (also known as Bluetooth Smart), Zigbee, or the like. The monitoring/communication interface component 1030 may further include one or more sensors such as a humidity sensor 1073 arranged to monitor ambient humidity conditions and/or a temperature sensor 1074 arranged to monitor ambient temperature conditions.

Continuing to refer to FIG. 10, the remote communication device 1086 may include a microprocessor 1080, a display 1081, a memory 1082, at least one user input element 1083, and (optionally) a camera or scanning device 1084 that may also provide barcode reading functionality. The remote communication device 1086 may be arranged to communicate (e.g., via a network 1095) with at least one other terminal or other remote device 1096 that may have a data storage element 1099 providing at least one of data storage and/or data retrieval capability.

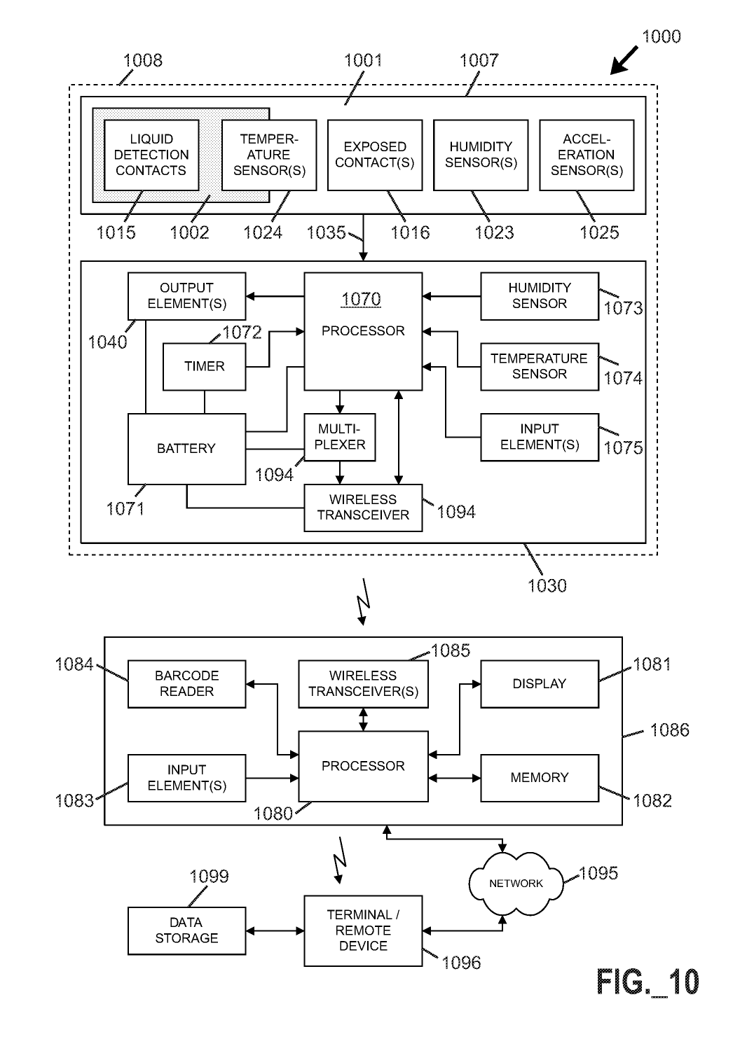

FIG. 11A-11B depict portions of a prototype apparatus for sensing a state of euhydration or dehydration of a mammalian subject, the apparatus including a substrate 1101 having arranged thereon a multi-dimensional array of electrical contacts 1115A-1115X (wherein the suffix “X” represents a variable and is not limited to any specific quantity or number of contacts) supporting a liquid collection element 1102, with the substrate 1101 including a curved extension 1101A supporting a flexible (e.g., ribbon-type) cable 1135 arranged to mate with a monitoring/communication interface component 1130 arranged to receive signals from the electrical contacts and interface with a computing and/or communication device (not shown) via a cable 1129. The curved extension portion 1101A is arranged to extend outside a mouth of a mammalian subject. The electrical contacts 1115A-1115X may be separated by a shared ground of neutral region 1118. A leading edge portion of the substrate 1101 defines a notch or recess 1113 arranged for placement under the tongue of a mammalian subject to receive at least a portion of the sublingual frenulum of the subject. Exposed electrical contacts 1116-1, 1116-2 are further provided proximate to a leading edge portion of the substrate 1101 and are not covered with the liquid collection element 1102 to permit the exposed contacts 1116-1, 1116-2 to detect insertion of at least a frontal portion of a sensing device in the mouth of a subject, and automatically establish a starting time (initial time value) and/or automatically start a timer indicative of the start of a sampling period, with an initial time value, elapsed time values, and/or values derived therefrom preferably being stored in a memory. In certain embodiments, the exposed contacts 1116-1, 1116-2 may also be used to determine state of the apparatus prior to insertion in the mouth of the mammalian subject, such as by performing a control check for the absence of saliva (e.g., detectable as the absence of closed contacts) prior to initiation of testing/sample collection. The substrate 1101 includes a raised registration feature 1119 arranged to receive a corresponding aperture or hole defined in the shared ground or neutral region 1118 to facilitate alignment and/or retention of the liquid collection element relative to the substrate 1101.

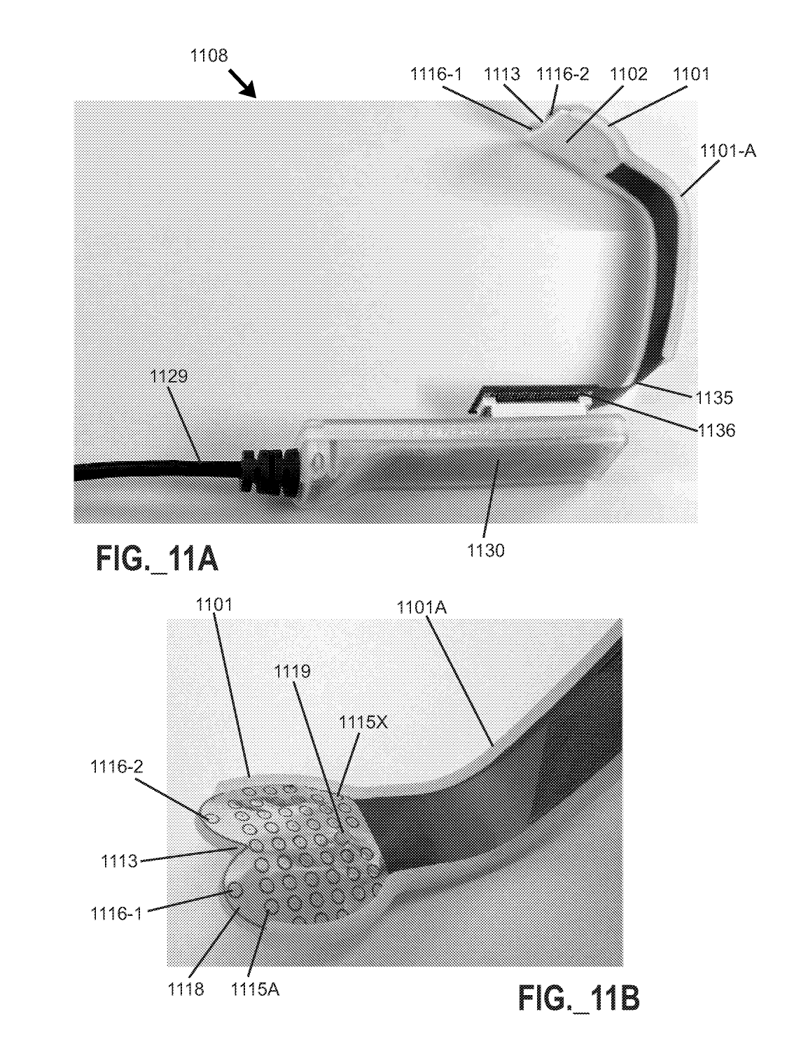

FIGS. 12A-12B depict a computer display including a software user interface representing the multi-dimensional array of electrical contacts 1215A-1215X and exposed contacts 1216-1 to 1216-2 of the prototype apparatus of FIG. 11A in first and second states of operation, respectively. The software user interface and display device may be associated with a remote communication device and/or other terminal or remote device. Darkened regions (e.g., circles) represent closed contacts (or reduction in electrical resistance or impedance), with darkness of each region increasing with elapsed time and/or relative to increased saturation due to saliva flow of mammalian subject since initiation of a sampling run. In addition to being dependent on degree of saturation, resistance or impedance values (and therefore darkness of the displayed regions) may also be affected by degree of conductivity of saliva. As shown in FIG. 12A, only one exposed contact 1216-1 is darkened, whereas in FIG. 12B (after additional elapsed time), both exposed contacts 1216-2 are darkened. The number of darkened circles (regions) corresponding to closed contacts is also increased in FIG. 12B relative to FIG. 12A, corresponding to increased area of saturation of portions of the liquid collection element and closure of an increased number of electrical contacts, wherein average darkness of shaded regions in FIG. 12B exceeds average darkness of shaded regions in FIG. 12A. In certain embodiments, state of individual temperature sensors or groups of temperature sensors of a multi-dimensional array of temperature sensors may be visually represented. In certain embodiments, data obtained from one sampling run may be visually compared to reference data, such as one or more raw or processed (e.g., averaged) sets of data corresponding to previously-captured sampling runs of the same mammalian subject, or one or more sets of raw or processed sets of data corresponding to one or more other mammalian subjects or groups of subjects.

FIG. 13 is a schematic side cross-sectional view of major salivary glands and selected elements in and around a human mouth, including the parotid gland 1, the submandibular gland 2, the sublingual gland 3, and minor glands 4 along the lower lip.

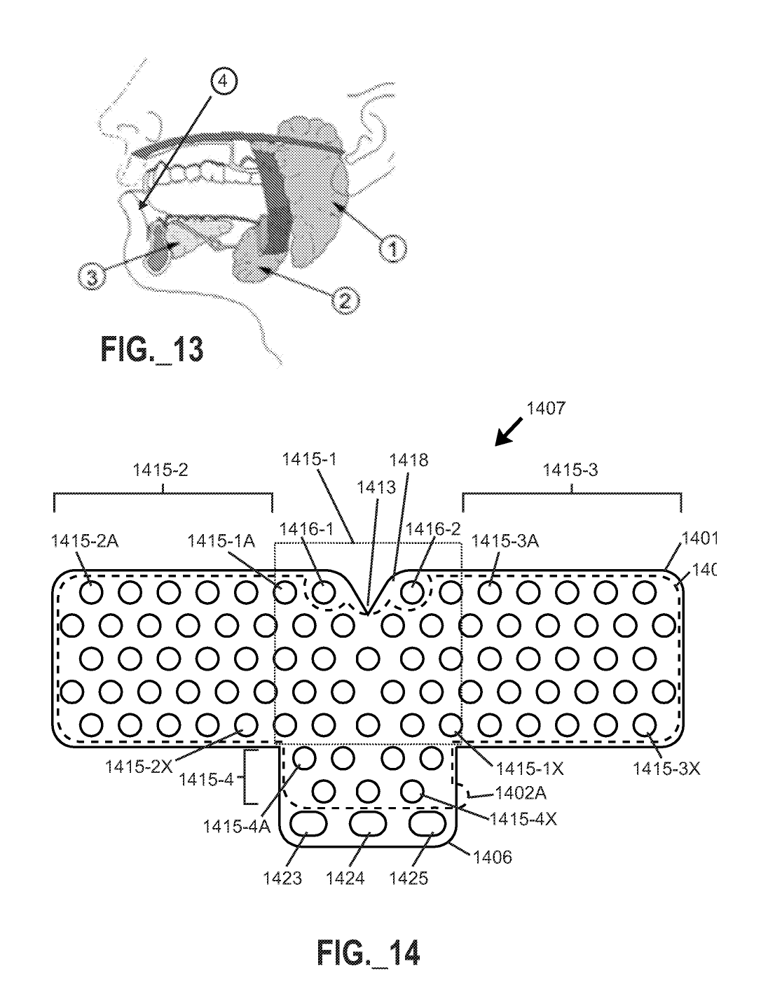

FIG. 14 illustrates a sampling component 1407 of an apparatus for sensing a state of euhydration, state of dehydration, or salivary secretion rate of a mammalian subject including a flexible substrate 1401 with a multi-dimensional array including multiple subsets of electrical contacts 1416-1A-1416-1X, 1416-2A-1416-2X, 1416-3A-1416-3X, 1416-4A-1416-4X, and supporting a liquid collection element 1402, wherein different regions 1415-1, 1415-2, 1415-3, 1415-4 of the substrate 1401 are arranged for placement in different locations of the mouth of the subject. The substrate 1401 defines a notch or recess 1413 along a leading edge thereof, with exposed contacts 1416-1, 1416-2 not being covered with the liquid collection element 1402. A first region 1415-1 of the substrate 1401 including contacts 1415-1A-1415-1X may be arranged for placement in a sublingual cavity of a subject. A second region 1415-2 of the substrate 1401 including contacts 1415-2A-1415-2X may be arranged for placement in a left buccal cavity of the subject. A third region 1415-3 of the substrate 1401 including contacts 1415-3A-1415-4X may be arranged for placement in a right buccal cavity of the subject. A fourth region 1415-4 of the substrate 1401 including contacts 1415-4A-1415-4X may be arranged for placement in a cavity between lower lip and teeth/gums of the subject. The sampling component may further include at least one humidity sensor 1423, at least one temperature sensor 1424, and/or at least one acceleration sensor 1425 (which may be arranged for placement inside or outside a mouth of a user). An end portion 1406 of the sampling component 1406 may include an interface cable (not shown) arranged to permit electrical communication with a monitoring/communication interface component (not shown). A protruding tab portion 1402A of the liquid collection element 1402 may embody a pull tab permitting grasping and facilitating peeling removal of the liquid collection element 1402 from the substrate 1401 (e.g., for weighing, for performance of one or more assays or other chemical tests using collected liquid, for retention in a suitable container, or for disposal). In certain embodiments, temperature sensors may be interspersed among the subsets of electrical contacts 1416-1A-1416-1X, 1416-2A-1416-2X, 1416-3A-1416-3X, 1416-4A-1416-4X and/or placed in a multi-dimensional array along an opposing side of the substrate, to permit sensing of temperature in different regions of a subject's mouth. In certain embodiments, sensing of temperature in different regions of a subject's mouth may be useful in determining proper alignment of apparatus within mouth. In certain embodiments, different (discrete) liquid collection elements may be provided over each subsets of electrical contacts 1416-1A-1416-1X, 1416-2A-1416-2X, 1416-3A-1416-3X, 1416-4A-1416-4X in the different regions 1415-1, 1415-2, 1415-3, 1415-4. Providing multiple subsets of electrical contacts 1416-1A-1416-1X, 1416-2A-1416-2X, 1416-3A-1416-3X, 1416-4A-1416-4X proximate to different salivary glands may permit function of different salivary glands of the same subject to be individually quantified and compared. The sampling component 1407 may be used in conjunction with a monitoring/communication interface component, a remote communication device, and interface software as described previously herein.

While the invention has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

1-52. (canceled)

53. An apparatus for sensing a state of euhydration, state of dehydration, or salivary secretion rate of a mammalian subject, the apparatus comprising:
a liquid collection element comprising a hydrophilic, porous medium or fibrous medium; and
a substrate including a frontal portion arranged to support the liquid collection element and including a leading edge defining a notch or recess, wherein the notch or recess is arranged to receive at least a portion of a sublingual frenulum of a mammalian subject upon insertion of the frontal portion under a tongue of the mammalian subject.

54. An apparatus according to claim 53, further comprising a plurality of saliva detection regions comprising a plurality of electrical contacts supported by the substrate, the plurality of electrical contacts being arranged to detect presence of saliva following migration of saliva through portions of the liquid collection element, wherein the plurality of saliva detection regions includes multiple non-collinearly arranged saliva detection regions arranged to detect a front of saliva advancing simultaneously in multiple directions.

55. An apparatus according to claim 54, further comprising at least one exposed electrical contact supported by the frontal portion of the substrate and arranged to detect a condition indicative of placement of the frontal portion into contact with at least one of oral mucosa and salivary duct secretions of the mammalian subject.

56. An apparatus according to claim 53, comprising a microprocessor in electrical communication with the plurality of electrical contacts and arranged to identify a state of euhydration, state of dehydration, or salivary secretion rate of the mammalian subject.

57. An apparatus for sensing a state of euhydration, state of dehydration, or salivary secretion rate of a mammalian subject, the apparatus comprising:
a liquid collection element comprising a hydrophilic, porous medium or fibrous medium arranged to contact oral mucosa of a mammalian subject;
a plurality of saliva detection regions comprising a plurality of electrical contacts arranged to detect presence of saliva following migration of saliva through portions of the liquid collection element, wherein the plurality of saliva detection regions includes multiple non-collinearly arranged saliva detection regions arranged to detect a front of saliva advancing simultaneously in multiple directions; and
a substrate arranged to support the plurality of electrical contacts and the liquid collection element.

58. An apparatus according to claim 57, wherein the multiple non-collinearly arranged saliva detection regions include multiple electrical contacts arranged in a multi-dimensional array.

59. An apparatus according to claim 57, wherein the multiple non-collinearly arranged saliva detection regions include multiple electrical contacts arrangeable along imaginary boundary lines of multiple concentric geometric shapes traceable over the substrate.

60. An apparatus according to claim 57, comprising a microprocessor in electrical communication with the plurality of electrical contacts and arranged to identify a state of euhydration, state of dehydration, or salivary secretion rate of the mammalian subject.

61. An apparatus according to claim 60, wherein the microprocessor is arranged to identify a state of euhydration, state of dehydration, or salivary secretion rate of the mammalian subject based at least in part on one of the following items (i) to (iv):
(i) a count of a number of electrical contacts triggered within a predetermined time period by saliva following migration of saliva through portions of the liquid collection element;
(ii) a rate of triggering of at least some electrical contacts of the plurality of electrical contacts;
(iii) a time within which a predetermined number of electrical contacts of the plurality of electrical contacts are triggered; and
(iv) a time until changes detected by one or more sensors ceases.

62. An apparatus according to claim 57, further comprising a signaling element in communication with the microprocessor and arranged to generate an output signal indicative of a state of euhydration, state of dehydration, or salivary secretion rate of the mammalian subject.

63. An apparatus according to claim 57, comprising a sampling portion and a monitoring portion, wherein the liquid collection element is arranged in the sampling portion, wherein each of the microprocessor and the signaling element is arranged in the monitoring portion, and wherein the sampling portion is operatively connected to the monitoring portion via at least one of an electrical communication cable, an electrical connector, and a wireless communication element.

64. An apparatus according to claim 57, wherein the substrate includes a frontal portion arranged to support the liquid collection element and includes a leading edge defining a notch or recess, wherein the notch or recess is arranged to receive at least a portion of a sublingual frenulum of the mammalian subject upon insertion of the frontal portion under a tongue of the mammalian subject.

65. An apparatus according to claim 57, further comprising at least one exposed electrical contact supported by the substrate and arranged to detect a condition indicative of placement of the substrate into contact with at least one of oral mucosa and salivary duct secretions of the mammalian subject.

66. An apparatus according to claim 57, further comprising a shared electrical ground region or shared electrically neutral region arranged between each electrical contact of the plurality of electrical contacts.

67. An apparatus according to claim 57, further comprising an accelerometer associated with the substrate and arranged to detect tilt or position of the substrate.

68. A method for sensing a state of euhydration, state of dehydration, or salivary secretion rate of a mammalian subject, the method comprising:
swabbing at least one portion of an oral cavity a mammalian subject with an absorptive medium to remove ambient saliva proximate to at least one salivary duct; and
following said swabbing, inserting into said at least one portion of the oral cavity, proximate to the at least one salivary duct, a liquid collection element comprising a hydrophilic, porous medium or fibrous medium supported by at least a frontal portion of a substrate, wherein the at least a frontal portion comprises a plurality of saliva detection regions including a plurality of electrical contacts arranged to detect presence of saliva following migration of saliva through portions of the liquid collection element, wherein the plurality of saliva detection regions includes multiple non-collinearly arranged saliva detection regions arranged to detect a front of saliva advancing simultaneously in multiple directions; and
using the plurality of electrical contacts to detect presence of saliva following migration of saliva through portions of the liquid collection element.

69. A method according to claim 68, further comprising utilizing at least one microprocessor in electrical communication with the plurality of electrical contacts to identify a state of euhydration, state of dehydration, or salivary secretion rate of the mammalian subject based at least in part on one of the following items (i) to (iv):
(i) a count of a number of electrical contacts triggered within a predetermined time period by saliva following migration of saliva through portions of the liquid collection element;
(ii) a rate of triggering of at least some electrical contacts of the plurality of electrical contacts;
(iii) a time within which a predetermined number of electrical contacts of the plurality of electrical contacts are triggered; and
(iv) a time until changes detected by one or more electrical contacts ceases; and
generating an output signal indicative of state of euhydration, state of dehydration, or salivary secretion rate of the mammalian subject.

70. A method according to claim 68, further comprising performing an assay utilizing saliva collected with the liquid collection element to generate a quantitative or qualitative output signal based on concentration of at least one analyte in the saliva.

71. A method according to claim 68, wherein the substrate comprises a flexible substrate; a first portion of the substrate includes a first subset of the plurality of electrical contacts; a second portion of the substrate includes a second subset of the plurality of electrical contacts; said inserting of the liquid collection element into the at least one portion of the oral cavity comprises inserting the first portion into a first location selected from: a sublingual cavity, a left buccal cavity, a right buccal cavity, and a cavity between lower lip and teeth/gums; said inserting of the liquid collection element into the at least one portion of the oral cavity comprises inserting the second portion into a second location selected from a sublingual cavity, a left buccal cavity, a right buccal cavity, and a cavity between lower lip and teeth/gums; wherein the second location differs from the first location; and wherein the method further comprises:
receiving signals from the plurality of electrical contacts; and
performing at least one of the following steps (i) and (ii):
(i) mapping salivary secretion rates in the first location and the second location; and
(ii) comparing salivary secretion rates in the first location and the second location.

72. A method according to claim 68, further comprising:
generating an output signal indicative of state of euhydration, state of dehydration, or salivary secretion rate of the mammalian subject; and
using the output signal to diagnose a disease state of the mammalian subject or to detect a side effect of an interaction of at least one drug with the mammalian subject.
